(19) United States
(12) Patent Application Publication
(10) Pub. No.: US 2018/0330044 A1
(43) Pub. Date: Nov. 15, 2018

(54) Methods Associated With A Database That Stores A Plurality Of Reference Genomes

(71) Applicant: Genome Research Limited, Cambridge (GB)

(72) Inventors: Trevor D. Lawley, London (GB); Hilary P. Browne, London (GB); Sam C. Forster, London (GB)

(73) Assignee: Genome Research Limited, Cambridge (GB)

(21) Appl. No.: 15/768,432

(22) PCT Filed: Oct. 14, 2016
(86) PCT No.: PCT/EP2016/074739

§ 371 Date: Apr. 13, 2018

(30) Foreign Application Priority Data
Oct. 16, 2015 (GB) 1518364.3

(51) Int. Cl. G06F 19/14; G06F 19/28; G06F 19/22

(52) U.S. Cl. CPC G06F 19/14; G06F 19/22; G06F 19/28

(57) ABSTRACT

Methods are provided of using a database that stores a plurality of reference genomes and phylogenetic information which relates the stored reference genomes to each other in a phylogenetic structure. These methods are useful in analysing the bacteria and/or bacterial lineages present in a sample and to identify a bacterium for use in therapy.

### FIELD OF THE INVENTION

The present invention relates (among other aspects) to methods associated with a database that stores a plurality of reference genomes and phylogenetic information which relates the stored reference genomes to each other in a phylogenetic structure.

### BACKGROUND

Over the past decade, the application of metagenome sequencing to elucidate the microbial composition and functional capacity present in the human microbiome has revolutionized many concepts in our basic biology. The growing availability of metagenomic sequencing and associated analysis tools has enabled quantification and understanding of this microbial diversity at a level not previously possible.

The importance of understanding the tremendous molecular and phenotypic diversity present within a single bacterial lineage has also only recently become evident. Whole genome phylogeny of hundreds of isolates from a single “species” has demonstrated each “species” represents an evolutionary web of highly related lineages with diverse phenotypic characteristics. For example, strains of many opportunistic pathogen species including Peptoclostridium difficile and Escherichia coli that cannot be differentiated by 16S rRNA gene profiling, can range from benign members of the gastrointestinal tract to highly virulent pathogens, inducing severe, sometimes fatal symptoms in the host.

There are two primary types of metagenomic sequencing currently performed, 16S profiling and whole genome or “shotgun” metagenomic sequencing. These are described below:

### 16S Profiling

In this technique, a variable region of the highly conserved 16S gene is amplified and the resulting product subjected to high throughput sequencing. The resulting short reads from the 16S gene (typically ˜100-150 base pairs) are then mapped (aligned) to a reference database using approaches such as the mothur pipeline (http://www.mothur.org/).

16S Profiling is highly efficient as all reads differentiate to the maximum level of phylogenetic resolution and this technique is standardized and widely used.

However, 16S profiling is limited to groups containing 16S gene (Bacteria and Archaea), and since the 16S gene is highly conserved, it is difficult to distinguish between different lineages at lower level branches of a phylogenetic tree. Resolution is therefore limited to distinguishing between groups (referred to as Operation Taxonomic Units, OTU) that differ in the small region of the 16S gene considered (typically Family or Genus level). That is, with 16S sequencing, deeper sequencing depth does not provide greater resolution.

Not all organisms have 16S gene, so these can't be identified by looking at the 16S gene and this, combined with bias potentially introduced by gene amplification, makes it difficult to determine the relative abundances of organisms present in a sample at a biologically meaningful level of resolution.

### Whole Genome Metagenomic Sequencing

Whole Genome metagenomic sequencing can be analyzed using de-novo assembly based approaches or using a lowest common ancestor approach. In this technique, the complete DNA sample (not just the 16S gene) is subjected to high throughput sequencing. The resulting short sequence reads (typically ˜100-150 base pairs)

Whole Genome Sequencing—De-Novo assembly

In De-novo assembly based approaches reads are “assembled” by looking for regions that correspond to overlapping reads

Advantageously, De-novo assembly does not rely on reference genomes, thus overcoming issues associated with culturing.

However, De-novo assembly suffers from limited resolution when two genomes from closely related species are considered. Also, there is an inability to define complete genomic units, since De-novo assembly is limited to regions of the genome that are sequenced.

De-novo assembly is also extremely computationally intensive (so impractical for large datasets), and requires substantial sequence coverage to provide a useable dataset.

### Whole Genome Sequencing—Lowest Common Ancestor Approach

In this technique, the short reads (typically ˜100-150 bp) are assigned based on known reference genomes. This approach is best described in the Kraken algorithm publication (PMID: 24580807).

Advantageously, the lowest common ancestor approach allows fast classification of organisms present within a sample, and has improved resolution compared to 16S or De-novo assembly.

However, the lowest common ancestor approach is dependent on the quality and coverage of reference genomes.

The present inventors have observed that the lowest common ancestor approach generally provides information regarding the number of reads mapped to reference genomes in a sample from which short reads have been obtained, rather than the relative abundances of the reference genomes in the sample, thus limiting resolution and the ability to compare between species.

### Characterisation of the Human Microbiome

Most of our understanding of the human microbiome and its role in health and disease has primarily been derived from culture independent, genomic approaches, as described above.

It is now appreciated that, for example, the composition of the human intestinal microbiota is important for providing resistance to pathogen invasion (referred to as ‘colonisation resistance’) (Lawley and Walker 2013) and that, if the microbiota is perturbed (also referred to as ‘dysbiosis’), the healthy base-line status can be restored through introduction of commensal intestinal bacteria (Petrof, Gloor et al. 2013, van Nood, Vrieze et al. 2013).

Attempts have been made to treat intestinal dysbiosis using faecal transplantation. Faecal transplantation involves transplanting intestinal bacteria from faeces of a healthy individual to an individual with an intestinal dysbiosis. This approach has been shown to provide an effective treatment for Clostridium difficile infection, for example (Petrof, Gloor et al. 2013, van Nood, Vrieze et al. 2013, Seekatz, Aas et al. 2014). However, faecal transplants have several drawbacks, including their undefined nature with respect to the bacteria and other microorganisms they contain, the availability of suitable donor material for large-scale clinical use, and administration of the faecal transplant to the patient. There thus remains a need in the art for defined bacterial mixtures for resolving dysbiosis and treatment of other diseases.

In order to utilise a bacterium that may be useful in resolving a dysbiosis or disease, the bacterium must first be isolated in culture, archived and characterised to ensure efficacy and safety. As the majority of the human microbiota is currently considered unculturable (Stewart 2012), this presents a significant limitation with regard to the bacteria which can be investigated and utilised as potential therapeutics.

One of the major limitations in culturing microbiota lies in characterising the bacteria present in a microbiota which have and/or have not been cultured using a particular set of culture conditions. Characterising bacteria successfully cultured using a set of culture conditions would allow the culture conditions to be used to prepare strain collections of the bacteria which could then be investigated for therapeutic applications. In addition, a means to identify bacteria not successfully cultured using a set of culture conditions would allow the culture conditions to be adjusted with a view to culturing bacteria of interest which were not successfully cultured initially. Methods for characterising the bacteria cultured from microbiota have been proposed (Goodman et al., 2011; US2014/045744). However, these methods rely on sequencing of the variable region 2 of the 16S ribosomal RNA (rRNA) gene and are thus not sufficiently sensitive to identify all of the species which were successfully isolated.

The present invention has been devised in light of the above considerations.

### SUMMARY OF THE INVENTION

A first aspect of the invention relates to using a database that stores a plurality of reference genomes and phylogenetic information which relates the stored reference genomes to each other in a phylogenetic structure.

For the purposes of this disclosure, a phylogenetic structure can be understood as a hierarchical structure which relates reference genomes to each other in one or more lineages, based on similarities/differences (e.g. genetic sequences that are present/not present) in the reference genomes. Computational techniques for inferring such a phylogenetic structure from stored reference genomes are well known, see e.g. Mauve (PMID: 15231754), Muscle (PMID: 15034147), and MAFFT (PMID: 23329690).

For the purpose of this disclosure, a lineage can be understood as a group of reference genomes inferred as being related to each other based on one or more similarities in the reference genomes (e.g. using a computational technique, as is known in the art).

In the phylogenetic structure, each lineage/reference genome may be related to one or more other lineages/reference genomes according to a parent-child relationship. For the avoidance of any doubt, a lineage can be parent to one or more other lineages in the phylogenetic structure (see e.g. FIG. 2(a) and FIG. 2(b)).

A visualisation of a very simple example phylogenetic structure shown in FIG. 2(b), where “LINEAGE BC” is parent to “GENOME B” and “GENOME C”, and “LINEAGE ABC” is parent to “GENOME A” and “LINEAGE BC”. A lineage can thus be visualised as a branch of a phylogenetic tree

The first aspect of the invention may provide:
A method of using a database that stores a plurality of reference genomes and phylogenetic information which relates the stored reference genomes to each other in a phylogenetic structure, the method including:
using a plurality of sequence reads obtained from a sample to count the number of sequence reads deemed to uniquely map to each of a plurality of lineages and/or reference genomes within the phylogenetic structure;
for each of the plurality of lineages and/or reference genomes to which at least one sequence read has been deemed uniquely mapped, normalizing the number of sequence reads counted as being deemed uniquely mapped to the lineage or reference genome using a measure that reflects the uniqueness of the lineage or reference genome so as to obtain an indication of the relative abundance of the lineage or reference genome within the sample.

Thus, according to this method, indications of the relative abundances of lineages and/or reference genomes within the sample can be obtained. As discussed in more detail below, such values can be very useful in a range of ‘downstream’ applications.

In some cases, the method may include using a plurality of sequence reads obtained from a sample to count the number of sequence reads deemed as being uniquely mapped to only a subset of lineages and/or reference genomes within the phylogenetic structure, e.g. where that subset of lineages and/or reference genomes corresponds only to lineages and/or reference genomes of interest for a particular experimental study.

However, preferably the method includes using a plurality of sequence reads obtained from a sample to count the number of sequence reads deemed as being uniquely mapped to a plurality of lineages and reference genomes (preferably all lineages and reference genomes) within the phylogenetic structure.

The method may include a preliminary step of inferring a phylogenetic structure from stored reference genomes, e.g. using a computational technique. As noted above, such computational techniques are well known, see e.g. Mauve (PMID: 15231754), Muscle (PMID: 15034147), and MAFFT (PMID: 23329690).

For the purposes of this disclosure, a measure of uniqueness of a lineage may be taken as a measure that reflects (e.g. by being proportional to) the combined length of one or more genetic sequences deemed to uniquely identify the lineage.

For the purposes of this disclosure, a measure of uniqueness of a reference genome may be taken as a measure that reflects (e.g. by being proportional to) the combined length of one or more genetic sequences deemed to uniquely identify the reference genome.

Preferably, the method includes, for each of the plurality of lineages and/or reference genomes, determining a measure that reflects the uniqueness of the lineage or reference genome (e.g. so that the resulting measures can be used in normalizing the number of sequence reads counted as being deemed uniquely mapped to the lineage or reference genome, as described above).

Preferably, the method includes, for each of the plurality of lineages and/or reference genomes, determining a measure that reflects the uniqueness of the lineage or reference genome (or a precursor of such a measure) by:
for the/each reference genome:
   identifying one or more genetic sequences deemed to uniquely identify the reference genome;
   determining a measure that reflects the uniqueness of the reference genome (or a precursor of such a measure) based on the (e.g. combined length of the) one or more genetic sequences deemed to uniquely identify the reference genome;
for the/each lineage:
   identifying one or more genetic sequences deemed to uniquely identify the lineage;
   determining a measure that reflects the uniqueness of the lineage (or a precursor of such a measure) based on the (e.g. combined length of the) one or more genetic sequences deemed to uniquely identify the lineage.

As discussed in more detail below, identifying one or more genetic sequences deemed to uniquely identify a reference genome/lineage can potentially be computationally intensive. Therefore, the method may include storing each measure that reflects the uniqueness of a lineage or reference genome (or precursor of such a measure) in the database (e.g. in a uniqueness field of the database, as described below). In this way, the measure that reflects the uniqueness of a lineage or reference genome can be obtained more quickly when a sample is analysed, without having to re-perform the potentially computationally intensive task of identifying one or more genetic sequences deemed to uniquely identify a reference genome/lineage.

Preferably, identifying one or more genetic sequences deemed to uniquely identify each of the plurality of lineages and/or reference genomes is performed based on a step of comparing each reference genome stored in the database with all other reference genomes stored in the database. This is preferably done before using a plurality of sequence reads obtained from a sample to count the number of sequence reads deemed to uniquely map to each of a plurality of lineages and/or reference genomes within the phylogenetic structure.

Identifying one or more genetic sequences deemed to uniquely identify each of the plurality of lineages and/or reference genomes may include:
comparing each reference genome stored in the database with all other reference genomes stored in the database;
for the/each reference genome:
   based on the comparison, identifying one or more genetic sequences that are deemed to uniquely identify the reference genome;
for the/each lineage:
   based on the comparison and the phylogenetic information, identifying one or more genetic sequences that are deemed to uniquely identify the lineage.

Comparing each reference genome stored in the database with all other reference genomes stored in the database preferably includes comparing each reference genome stored in the database with the majority of the genetic content of (preferably 90% of the genetic content of, preferably the entirety of the genetic content of) all other reference genomes stored in the database. In this way, it is possible to identify one or more genetic sequences that are deemed to uniquely identify a reference genome or lineage, even when that reference genome is very closely relate to other reference genomes/lineages in the database.

In contrast, many current comparison methods use small “marker sequences” that may represent less than 1% of genetic content within reference genomes.

More preferably, identifying one or more genetic sequences deemed to uniquely identify each of the plurality of lineages and/or reference genomes includes, for each reference genome in the database:
defining a plurality of segments, each segment containing a different genetic sequence from the reference genome;
for the genetic sequence contained in each segment:
   comparing the genetic sequence contained in the segment with (preferably the majority of the genetic content of, preferably 90% of the genetic content of, preferably the entirety of the genetic content of) all other reference genomes in the database to establish whether the segment maps to any of the other reference genomes;
   if the genetic sequence contained in the segment maps to no other reference genome in the database, identifying the genetic sequence contained in the segment as being deemed to uniquely identify the reference genome;
   if the genetic sequence contained in the segment maps to one or more other reference genomes in the database, and if it is determined using the phylogenetic information that the genetic sequence contained in the segment maps to at least a majority of the reference genomes in a lineage (preferably maps to 90% or more of the reference genomes in a lineage, more preferably maps to all of the reference genomes in a lineage, more preferably maps to all of the reference genomes in a lineage and to no other reference genomes in the database), identifying the genetic sequence contained in the segment as being deemed to uniquely identify the lineage.

Although potentially computationally intensive, comparing the genetic sequence contained in a segment with the majority of the genetic content of (preferably 90% of the genetic content of, preferably the entirety of the genetic content of) all other reference genomes in the database to establish whether the segment maps to any of the other reference genomes is preferred, since this help to maximise resolution, i.e. helps to allow the identification of one or more genetic sequences that are deemed to uniquely identify closely related reference genomes and lineages.

Preferably, identifying one or more genetic sequences deemed to uniquely identify each of the plurality of lineages and/or reference genomes is performed before the sequence reads are obtained from the sample, since these steps can be computationally intensive and do not require the sequence reads obtained from the sample in order to be performed.

Preferably, identifying one or more genetic sequences deemed to uniquely identify each of the plurality of lineages and/or reference genomes is performed each time a new reference genome is stored in the database, since adding a new reference genome to the database may cause a change in the genetic sequences identified as being deemed to uniquely identify a lineage/reference genome.

The plurality of segments defined for each reference genome may have a predetermined length, and preferably include each possible segment of that length that could be defined for the reference genome. The plurality of segments could be obtained using a sliding window technique, e.g. in which a window of predetermined length (e.g. 100 base pairs) is aligned with the start of the reference genome to define a first segment, and then the window is moved along the reference genome by a single base pair at a time to define further segments until each possible segment has been defined for the reference genome. Sliding window techniques are well known in the art.

The predetermined length of the segments may be chosen based on practical considerations, e.g. based on computational power/time required to perform calculations. Preferably, the predetermined length of the segments is chosen to be the same as the length of the sequence reads obtained from the sample (discussed below). However, the predetermined length of the segments could be selected from a wide range, e.g. 50-10,000+ base pairs.

Comparing the genetic sequence contained in a segment with all other reference genomes may be performed with an aligner, as is known in the art. In the examples below, a bowtie2 read aligner is used (see e.g. PMID: 22388286), though there are many other aligners that could be used such as bwa (see e.g. PMID 19451168).

For the avoidance of any doubt, a segment need not be identical to a reference genome in order for that sequence read to be established as being “mapped” to that reference genome, since it is known in the art that reference genomes may contain inaccuracies, e.g. introduced by the sequencing technologies used to derive the genetic sequences contained therein. Accordingly, any algorithm/aligner used to establish whether any segments map to a reference genome may be configured to ignore minor differences (e.g. differences of 2-3 base pairs could be ignored for a segment 100 base pairs in length). As would be appreciated by a skilled person, ignoring minor differences in this way may result in minor differences caused by real mutations to be overlooked (i.e. so overlooked minor differences might not be limited to minor differences introduced by sequencing technologies).

Techniques for identifying one or more genetic sequences deemed to uniquely identify a lineage or reference genome other than those described above could be envisaged by a skilled person.

Using a plurality of sequence reads obtained from a sample to count the number of sequence reads deemed to uniquely map to each of a plurality of lineages and/or reference genomes within the phylogenetic structure may include:
for the/each reference genome:
   comparing the plurality of sequence reads with the reference genome to establish whether any sequence reads map to the reference genome and to no other reference genome stored in the database;
   if a sequence read maps to the reference genome and to no other reference genome stored in the database, counting the sequence read as being deemed to uniquely map to the lineage;
for the/each lineage:
   comparing the plurality of sequence reads with one or more genetic sequences deemed to uniquely identify the lineage to establish whether any sequence reads map to any of the identified one or more genetic sequences;
   if a sequence read maps to any of the one or more genetic sequences deemed to uniquely identify the lineage, counting the sequence read as being deemed to uniquely map to the lineage.

Other ways to count the number of sequence reads deemed to uniquely map to each of a plurality of lineages and/or reference genomes within the phylogenetic structure could be envisaged by a skilled person. For example, a sequence read could be deemed to uniquely map to a lineage if the sequence read maps to more than one reference genome in the database, and if it is determined using the phylogenetic information that the sequence read maps to at least a majority of the reference genomes in a lineage (preferably maps to 90% or more of the reference genomes in a lineage, more preferably maps to all of the reference genomes in a lineage, more preferably maps to all of the reference genomes in a lineage and to no other reference genomes in the database).

Comparing the genetic sequence contained in a sequence read with all other reference genomes may be performed with an aligner, as is known in the art. In the example below, a bowtie2 read aligner is used (see e.g. PMID: 22388286), though there are many other aligners that could be used such as bwa (see e.g. PMID 19451168).

Preferably, comparing the plurality of sequence reads with each reference genome includes comparing each sequence read with the majority of the genetic content of (preferably 90% of the genetic content of, preferably the entirety of the genetic content of) each reference genome. This allows more sequence reads to be uniquely mapped to reference genomes, compared with methods in which the minority of genetic content of the reference genomes are used. In contrast, many current comparison methods use small “marker sequences” that may represent less than 1% of genetic content within reference genomes.

Techniques for counting the number of sequence reads deemed to uniquely map to each of a plurality of lineages and/or reference genomes within a phylogenetic structure are known in the art (see e.g. PMID: 24580807).

For the avoidance of any doubt, a sequence read need not be identical to a genetic sequence deemed to uniquely identify a lineage in order for that sequence read to be established as being “mapped” to that genetic sequence, since it is known in the art that the sequence reads and genetic sequences deemed to uniquely identify a lineage may contain inaccuracies, e.g. introduced by the sequencing technologies used to derive the genetic sequences contained therein. Accordingly, any algorithm/aligner used to establish whether any sequence reads map to any of the one or more genetic sequences deemed to uniquely identify the lineage may be configured to ignore minor differences (e.g. for a sequence read 100 base pairs in length, differences of 2-3 base pairs could be ignored). As would be appreciated by a skilled person, ignoring minor differences in this way may result in minor differences caused by real mutations to be overlooked (i.e. overlooked minor differences might not be limited to minor differences introduced by sequencing technologies).

Similarly, a sequence read need not be identical to a reference genome in order for that sequence read to be established as being “mapped” to that reference genome, since it is known in the art that the sequence reads and reference genomes may contain inaccuracies, e.g. introduced by the sequencing technologies used to derive the genetic sequences contained therein. Accordingly, any algorithm/aligner used to establish whether any sequence reads map to a reference genome may be configured to ignore minor differences (e.g. for a sequence read 100 base pairs in length, differences of 2-3 base pairs could be ignored). Again, as would be appreciated by a skilled person, ignoring minor differences in this way may result in minor differences caused by real mutations to be overlooked (i.e. overlooked minor differences might not be limited to minor differences introduced by sequencing technologies).

Example techniques for identifying one or more genetic sequences deemed to uniquely identify at least one lineage (preferably a plurality of lineages, more preferably all lineages) within the phylogenetic structure have already been discussed above. Also see e.g. PMID: 24580807.

Normalizing the number of sequence reads that were counted as being uniquely mapped to a lineage or reference genome of interest using a measure that reflects the uniqueness of that lineage or reference genome may simply involve dividing the counted number of sequence reads by the measure.

Preferably, the database includes an entry for each reference genome and each lineage within the phylogenetic structure.

Preferably, the entry for each reference genome includes a reference genome field for storing the reference genome or a pointer to the reference genome.

Preferably, the entry for each lineage/reference genome includes a parent field for storing a pointer to a parent of the lineage/reference genome within the phylogenetic structure.

Preferably, the entry for each lineage/reference genome includes a uniqueness field for storing a measure that reflects the uniqueness of the lineage or reference genome or a precursor of such a measure, which may have been determined as described above. As noted above, the measure or precursor stored in this field may allow the measure that reflects the uniqueness of the lineage or reference genome to be obtained more quickly when a sample is analysed, without having to re-perform the potentially computationally intensive task of identifying one or more genetic sequences deemed to uniquely identify a reference genome/lineage.

Since uniqueness can change when a new reference genome is stored in the database, the uniqueness field is preferably recalculated each time a new reference genome is stored in the database.

The method may include obtaining the plurality of sequence reads from the sample, e.g. using a DNA sequencer.

Preferably, the sequence reads are obtained by a shotgun sequencing process, in which the DNA contained in the sample is broken up randomly into small segments which are then sequenced to obtain the plurality of sequence reads.

Preferably, the plurality of sequence reads from the sample are obtained from across the complete DNA of organisms within the sample (e.g. not just the 16S gene), e.g. whole genome shotgun sequencing.

The number of sequence reads obtained may be chosen using a measure that reflects the uniqueness of a lineage or reference genome of interest (e.g. determined as indicated above).

For example, the number of sequence reads to be obtained may be chosen to be at least m/u, where m is a minimum number of reads deemed appropriate for confident detection of a lineage or reference genome of interest, and where u is the internal uniqueness for that lineage or reference genome of interest, which represents the proportion of that individual lineage or reference genome of interest that is unique (relative to the genetic content of the individual lineage or reference genome). For a typical experiment, m is preferably 100 or more, more preferably 1000 or more.

The length of the sequence reads is preferably high enough to allow the sequence reads to be uniquely mapped to reference genomes in the database whilst being low enough to allow the sequence reads to be obtained with a high throughput.

Preferably, the sequence reads each have a length of at least 35 base pairs, more preferably 80 or more base pairs, so that random sequence reads can uniquely identify a reference genome. 100-150 base pairs would be typical with existing technologies. However, other lengths are plausible, and future sequencing technologies may result in other lengths becoming preferred.

The sample may be prepared to be suitable for DNA sequencing according to standard methods, known in the art.

In some embodiments, the reference genomes stored in the database may be (or may include) bacterial reference genomes.

The first aspect of the invention may provide an apparatus configured to perform a method as set out above.

The apparatus may include a computer configured (e.g. programmed) to perform a method as set out above (and/or any combination of steps set out above that involve manipulation of data).

The first aspect of the invention may provide a computer-readable medium having computer-executable instructions configured to cause a computer to perform a method as set out above (and/or any combination of steps set out above that involve manipulation of data).

A second aspect of the invention may provide methods which utilise a method according to the first aspect of the invention.

By way of example, the first aspect of the invention may find utility in the analysis of bacteria and/or bacterial lineages present in a sample, the analysis of bacteria and/or bacterial lineages which have or have not been cultured using a microbial culturing method, methods of preparing culture collections of bacteria of interest, and methods of obtaining genomic sequences of bacteria of interest.

In addition, the first aspect of the invention may find utility in identifying therapeutic bacteria, and in the diagnosis of diseases characterised by the presence of a bacterium.

These and other aspects are described below.

A sample, as referred to herein, may be a sample obtained from any source which is expected to comprise a microorganism, such as a bacterium. The sample may thus be a sample comprising a microorganism, e.g. a bacterium. Samples comprising microorganisms, including bacteria, can be obtained from many sources, including humans, animals, and environmental sources, such as soil samples.

In a preferred embodiment, the sample is obtained from an individual, i.e. a human individual. In this case, the sample may be a microbiota sample. Microbiota in this context refers to the microorganisms that are present on and in an individual. For example, intestinal microbiota and skin microbiota refer to the microbiota present in the intestine and on the skin of an individual, respectively.

The individual from whom a sample has been obtained may be, for example, a healthy individual or an individual with a disease or dysbiosis, as applicable. Dysbiosis may refer to an imbalance in the microbiota of an individual, and has been implicated in a number of diseases and disorders, such as inflammatory bowel disease (IBD).

The sample may be a body fluid or solid matter, or tissue biopsy, such as a faecal sample, a urine sample, a skin scrape, a colon biopsy, a lung biopsy, or a skin biopsy. In one example, the sample may be a faecal sample.

Where the context requires, the sample may be an uncultured sample, i.e. a sample which has not been subjected to any culturing, such as bacterial culturing. This is important in the context of identifying microorganisms, such as bacteria, present in the microbiota of an individual, for example.

For the purpose of this disclosure, a bacterial lineage, which is present e.g. in a sample, can be understood as a group of bacteria with genomes inferred as being related to each other based on one or more similarities in the genomes (e.g. using a computational technique, as is known in the art).

The second aspect of the invention may provide:
A method of analysing the bacteria and/or bacterial lineages present in a sample, wherein the method includes:
obtaining a plurality of sequence reads from (e.g. by performing whole genome shotgun sequencing of DNA extracted from) (i) a first portion of a sample obtained from an individual and using a method according to the first aspect of the invention to obtain indications of the relative abundances of one or more lineages and/or reference genomes within the first portion of the sample;
obtaining a plurality of sequence reads from (e.g. by performing whole genome shotgun sequencing of DNA extracted from) (ii) bacteria cultured from a second portion of the sample using a bacterial culturing method and using a method according to the first aspect of the invention to obtain indications of the relative abundances of one or more lineages and/or reference genomes within the cultured portion of the sample; and
comparing the indications of the relative abundances of the lineages and/or reference genomes within the first portion of the sample with the indications of the relative abundance of the lineages and/or reference genomes within the cultured sample.

Methods for obtaining a plurality of sequence reads, such as whole genome shotgun sequencing, are known in the art and are described elsewhere herein. Methods for extracting DNA from a sample are similarly known.

The method according to the second aspect of the invention may be a method of determining the bacteria and/or bacterial lineages present in a sample obtained from an individual which have or have not been cultured using a bacterial culturing method, wherein the method includes:
determining the bacteria and/or bacterial lineages present in the first portion of the sample which were and/or were not cultured using the bacterial culturing method by comparing the indications of the relative abundances of the lineages and/or reference genomes within the first portion of sample with the indications of the relative abundance of the lineages and/or reference genomes within the cultured sample.

This may find application, for example, in determining the proportion (e.g. percentage) of bacteria and/or bacterial lineages present in a sample, such as a microbiota sample obtained from an individual which have or have not been cultured using a bacterial culturing method. This may be useful, for example, when formulating or developing a broad range medium for culturing bacteria from a sample, such as a microbiota sample, or comparing different broad range media with respect to the proportion (e.g. percentage) of bacteria from a sample, such as a microbiota sample, whose growth the medium can support.

A method according to the second aspect may thus be a method of determining the proportion (e.g. percentage) of bacteria present in a sample, such as a sample obtained from an individual, which have or have not been cultured using a bacterial culturing method, wherein the method includes:
determining the proportion (e.g. percentage) of bacteria and/or bacterial lineages present in the first portion of the sample which were and/or were not cultured using the bacterial culturing method by comparing the indications of the relative abundances of the lineages and/or reference genomes within the first portion of sample with the indications of the relative abundance of the lineages and/or reference genomes within the cultured sample.

A method according to the second aspect may be used in the context of determining the bacteria which have been cultured using multiple, alternate, bacterial culturing methods. An alternate bacterial culturing method in this context refers to a different bacterial culturing method. The bacterial culturing methods may either be performed in parallel, using multiple portions of the same sample, or sequentially using different samples. In the latter case, the samples are preferably obtained from the same source, for example the same individual, to allow comparison between the bacteria successfully cultured using each culturing method. Alternate bacterial culturing methods may be used as part of a process for culturing the majority of the bacteria present in a sample, as different bacteria present in a sample are likely to have different growth requirements.

Where the bacterial culturing methods are employed sequentially, this may be as part of an iterative process for culturing the majority of the bacteria present in a sample, in which the bacteria and/or bacterial lineages present in a first sample which were and/or were not cultured using a first bacterial culturing method are determined, followed by determining the bacteria and/or bacterial lineages present in a second sample, preferably obtained from the same source at the first sample, e.g. the same individual, which were and/or were not cultured using a second, alternate, bacterial culturing method, and optionally determining the bacteria and/or bacterial lineages present in a second portion of the sample which were cultured using the second bacterial culturing method but which were not cultured using the first bacterial culturing method. This process can be repeated until the majority of the bacteria present in the sample have been cultured. This is useful, for example, for preparing culture collections of bacteria reflecting the majority of the bacteria present in a microbiome obtained from an individual, e.g. a healthy individual, or obtaining genomic sequences of the majority of the bacteria present in a microbiome obtained from a healthy individual and/or an individual with a dysbiosis.

A “majority” in this context may refer to culturing of at least 70%, at least 75%, at least 80%, at least 85%, at least 90%, at least 91%, at least 92%, at least 93%, at least 94%, at least 95%, at least 96%, at least 97%, at least 98%, or at least 99% of the bacterial present in a sample. “Bacteria” in this context may refer to a bacterial genus or a bacterial species. The present inventors have shown, for example, that using the broad range medium YCFA, with and without prior ethanol treatment and addition of taurocholate, bacteria from 97% of the most abundant genera and 90% of the most abundant species detected in a sample using metagenomics shotgun sequencing could be isolated. It is expected that reiteration of the culturing step using an alternate medium would allow an even higher proportion (e.g. percentage) of the bacterial genera and bacterial species present in a sample to be cultured.

The method according to the second aspect of the invention may therefore further comprise:
obtaining a plurality of sequence reads from (e.g. by performing whole genome shotgun sequencing of DNA extracted from) (iii) bacteria cultured from a second sample, preferably obtained from the same source, e.g. the same individual, as the sample in (i) and (ii), using an alternate bacterial culturing method and using a method according to the first aspect to obtain indications of the relative abundances one or more lineages and/or reference genomes within the alternately cultured sample; and
comparing the indications of the relative abundances of the lineages and/or reference genomes within the alternately cultured sample with the indications of the relative abundance of the lineages and/or reference genomes within the first portion of the sample and, optionally, the indications of the relative abundance of the lineages and/or reference genomes within the cultured sample.

Alternatively, where alternate bacterial culturing methods are used sequentially, a method according to the second aspect of the invention may comprise:
obtaining a plurality of sequence reads from (e.g. by performing whole genome shotgun sequencing of DNA extracted from) (iii) bacteria cultured from a third portion of the sample using an alternate bacterial culturing method and using a method according to the first aspect to obtain indications of the relative abundances one or more lineages and/or reference genomes within the alternately cultured sample; and
comparing the indications of the relative abundances of the lineages and/or reference genomes within the alternately cultured sample with the indications of the relative abundance of the lineages and/or reference genomes within the first portion of the sample and, optionally, the indications of the relative abundance of the lineages and/or reference genomes within the cultured sample.

In this case, the method according to the second aspect may be a method of determining the bacteria and/or bacterial lineages present in a sample obtained from an individual which have been cultured using an alternate bacterial culturing method, wherein the method includes:
(a) determining the bacteria and/or bacterial lineages present in the sample which were and/or were not cultured using the alternate bacterial culturing method by comparing the indications of the relative abundances of the lineages and/or reference genomes within the alternately cultured sample with the indications of the relative abundance of the lineages and/or reference genomes within the first portion of the sample; or
(b) determining the bacteria and/or bacterial lineages present in the sample which were cultured using the alternate bacterial culturing method and which were not cultured with the bacterial culturing method by comparing the indications of the relative abundances of the lineages and/or reference genomes within the alternately cultured sample with the indications of the relative abundance of the lineages and/or reference genomes within the first portion of the sample and the relative abundance of the lineages and/or reference genomes within cultured sample.

An alternate bacterial culturing method may be specifically adapted for, or specifically selected for, culturing bacteria which were not cultured with a first bacterial culturing method. Bacterial culturing methods for many bacterial families, genera and species are known in the art, as are methods for adapting a bacterial culturing method to culture bacteria from a particular bacterial family, genus, or species of interest. Similarly, many bacterial culturing methods, and methods for adapting bacterial culturing methods, to culture bacteria with a particular genotype and/or phenotype of interest are known. By identifying one or more bacteria of interest which were not cultured using a bacterial culturing method, thus allows a bacterial culturing method to be selected for, or the bacterial culturing method adapted for, culturing said bacteria of interest. Again this is useful, for example, for preparing culture collections of bacteria reflecting the majority of the bacteria present in a microbiome obtained from an individual, e.g. a healthy individual, or obtaining genomic sequences of the majority of the bacteria present in a microbiome obtained from a healthy individual and/or an individual with a dysbiosis.

Culture collections of bacteria of interest are useful for a number of different applications. For example, culture collections representing bacteria from the human microbiome may serve as a repository of potential candidates for bacteriotherapy of a disease or dysbiosis

A method according to the second aspect may thus be a method of preparing a culture collection of bacteria of interest present in a sample obtained from an individual, the method including identifying a bacterial culturing method for culturing the bacteria of interest, wherein the method includes:
determining the bacteria of interest present in the sample which were cultured using the bacterial culturing method by comparing the indications of the relative abundances of the lineages and/or reference genomes within the first portion of sample with the indications of the relative abundance of the lineages and/or reference genomes within the cultured sample; and
employing the bacterial culturing method to prepare a collection of cultures of said bacteria of interest from the sample.

The cultures are preferably pure cultures of bacteria. A pure culture may be a culture of a single bacterium.

Many bioinformatics approaches require the genomic sequence of a bacterium of interest to be known. For example, genomic sequences of bacteria can be compiled into databases which can then interrogated. Methods for whole genome sequencing are known in the art.

A method according to the second aspect of the invention may therefore be a method of obtaining the genomic sequence of one or more bacteria of interest present in a sample obtained from an individual, wherein the method includes:
determining the bacteria of interest present in the sample which were cultured using the bacterial culturing method by comparing the indications of the relative abundances of the lineages and/or reference genomes within the first portion of sample with the indications of the relative abundance of the lineages and/or reference genomes within the cultured sample;
employing the bacterial culturing method to prepare cultures of one or more of the bacteria of interest from the sample, and
determining the genomic sequence(s) of said bacteria.

The genomic sequence(s) of one or more bacteria of interest obtained using a method of the invention may be stored (e.g. as a new reference genome) in a database that stores reference genomes (e.g. a reference database as described above). Thus, the coverage of a database that stores reference genomes (e.g. a reference database as described above) can be improved by the methods described herein. As noted below, the methods according to the first aspect may be found particularly effective when used with a database that provides thorough genome coverage in an area of interest (e.g. an area which reflects the expected content of a sample of interest).

The worldwide emergence of bacterial resistance to antibacterial agents has produced a need for new methods of combating bacterial infections. The use of (harmless) bacteria to displace or inhibit pathogenic bacteria, such as Clostridium difficile, has been investigated for this purpose.

In addition, it is now thought that dysbiosis plays a role in a number of diseases, including inflammatory bowel disease. The administration of (harmless bacteria, e.g. through faecal transplanatation, represents a promising approach for treating (resolving) dysbiosis. However, treatment regimens such a faecal transplantation have a number of disadvantages, including their undefined nature with respect to the bacteria and other microorganisms they contain, the availability of suitable donor material for large-scale clinical use, and administration of the faecal transplant to the patient.

There thus remains a need in the art for identifying bacteria which can be used in the treatment of diseases characterised by the presence of a pathogenic bacterium and for the treatment of dysbiosis.

The second aspect of the invention may therefore provide:
A method of identifying a bacterium for bacteriotherapy for a dysbiosis, the method comprising:
obtaining a plurality of sequence reads from (e.g. by performing whole genome shotgun sequencing of DNA extracted from) a sample obtained from a patient with the dysbiosis;
using a method according to the first aspect of the invention to obtain indications of the relative abundances of lineages and/or reference genomes within the sample;
comparing the indications of the relative abundances of the lineages and/or reference genomes within the sample with the relative abundance of the lineages and/or reference genomes in a control;
selecting a bacterium with a genome, or belonging to a lineage, which is absent from the sample obtained from the patient but present in the control, or which is present at a lower relative abundance in the sample obtained from the patient compared with the control, for bacteriotherapy for the dysbiosis.

A patient as referred to herein is preferably a human patient.

A lower relative abundance of a bacterium may refer to a relative abundance which less than 50%, less than 40%, less than 30%, less than 20%, less than 10%, less than 5%, less than 4%, less than 3%, less than 2%, or less than 1% of the relative abundance of the bacterium in the control.

Alternatively, a lower relative abundance of a bacterium may refer to a relative abundance which is 2-fold or more, 5-fold or more, 10-fold or more, 20-fold or more, 30-fold or more, 40-fold or more, 50-fold or more, or 100-fold or more lower than the relative abundance of the bacterium in the control.

The control in this context may be a sample obtained from an individual without the dysbiosis, e.g. a healthy individual, or a group of such individuals. Alternatively, the control may be a reference value for the expected abundance of the bacterium in an individual without the dysbiosis.

A dysbiosis may refer to an imbalance in the microbiota of an individual. An imbalance in this context may refer to a disruption in the normal diversity and/or function of the microbiota. For example, dysbiosis may refer to an imbalance in, such as disruption in the normal diversity and/or function of, the commensal bacteria of an individual. A dysbiosis may be associated with one or more (disease) symptoms or may be symptomless.

Dysbiosis is thought to play a role in a number of diseases and syndromes, including: inflammatory bowel disease (IBD) (such as Crohn's Disease and ulcerative colitis); cancer (including colorectal cancer); enteric microbial infections, such as enteric bacterial infections (including Clostridium difficile infections), enteric viral infections, or enteric fungal infections; hepatic encephalopathy; asthma; Parkinson's disease, multiple sclerosis, autism, irritable bowel syndrome (IBS), coeliac disease, allergies, metabolic syndrome, cardiovascular disease, and obesity.

The second aspect of the invention may also provide:
A method of identifying a bacterium for bacteriotherapy for a dysbiosis, the method comprising:
obtaining a plurality of sequence reads from (e.g. by performing whole genome shotgun sequencing of DNA extracted from) (i) a first sample obtained from a patient with the dysbiosis;
obtaining a plurality of sequence reads from (e.g. by performing whole genome shotgun sequencing of DNA extracted from) (ii) a second sample obtained from the same patient after the patient has received a faecal transplant;
using a method according to the first aspect of the invention to obtain indications of the relative abundances of lineages and/or reference genomes within the sample;
comparing the indications of the relative abundances of the lineages and/or reference genomes within the first sample with the relative abundance of the lineages and/or reference genomes in the second sample;
selecting a bacterium with a genome, or belonging to lineage, which is absent from the first sample but present in the second sample, or which is present at a lower relative abundance in the first sample compared with the second sample, for bacteriotherapy for the dysbiosis.

Methods for faecal transplantation are known in the art. The faecal transplant is a faecal transplant from an individual without the dysbiosis, e.g. a healthy individual.

The second aspect of the invention may also provide:
A method of identifying a bacterium for therapy of a disease characterised by the presence of a pathogenic bacterium, the method comprising:
obtaining a plurality of sequence reads from (e.g. by performing whole genome shotgun sequencing of DNA extracted from) (i) a first sample obtained from a first asymptomatic carrier of the pathogenic bacterium, and obtaining a plurality of sequence reads from (e.g. by performing whole genome shotgun sequencing of DNA extracted from) (ii) a second sample obtained from a second asymptomatic carrier of the pathogenic bacterium;
using a method according to the first aspect, to obtain indications of the relative abundances of lineages and/or reference genomes within the first sample and the second sample;
comparing the indications of the relative abundances of the lineages and/or reference genomes within the first sample with the relative abundance of the lineages and/or reference genomes in the second sample;
selecting a bacterium with a genome, or belonging to a lineage, which is common to the first and second sample for bacteriotherapy for the disease.

A bacterium which is common to the first and second samples, as referred to above, may be present in the first and second samples at the same, or substantially the same, abundance.

An asymptomatic carrier in this context may refer to an individual who is infected with a pathogenic bacterium but exhibits no disease symptoms normally associated with the pathogenic bacterium.

The second aspect of the invention may also provide:
A method of identifying a bacterium for therapy of a disease characterised by the presence of a pathogenic bacterium, the method comprising:
   obtaining a plurality of sequence reads from (e.g. by performing whole genome shotgun sequencing of DNA extracted from) (i) a first sample obtained from an asymptomatic carrier of the pathogenic bacterium, and obtaining a plurality of sequence reads from (e.g. by performing whole genome shotgun sequencing of DNA extracted from) (ii) a second sample obtained from healthy individual;
using a method according to the first aspect to obtain indications of the relative abundances of lineages and/or reference genomes within the first sample and the second sample;
comparing the indications of the relative abundances of the lineages and/or reference genomes within the first sample with the relative abundance of the lineages and/or reference genomes in the second sample;
selecting a bacterium with a genome, or belonging to a lineage, which is present in the first sample but absent from the second sample, or which is present at a higher relative abundance in the first sample compared with the second sample, for bacteriotherapy for the disease.

A higher relative abundance of a bacterium may refer to a relative abundance which is 50% or more, 100% or more, 500% or more, or 1000% or more higher than the relative abundance of the bacterium in the second sample.

Alternatively, a higher relative abundance of a bacterium may refer to a relative abundance which is 2-fold or more, 5-fold or more, 10-fold or more, 20-fold or more, 30-fold or more, 40-fold or more, 50-fold or more, 100-fold or, 500-fold or more, or 100-fold or more higher than the relative abundance of the bacterium in the second sample.

Many disease characterised by the presence of pathogenic bacteria are known in the art and include Clostridium difficile infection, and methicillin-resistant Staphylococcus aureus (MRSA) infection.

In addition, the second aspect of the invention may provide:
A bacterium identified using a method of identifying a bacterium for bacteriotherapy for a dysbiosis or disease as described above, wherein the bacterium is for use in a method of treating the dysbiosis or disease in an individual.

The second aspect of the invention may also provide:
A bacterium for use in a method of treating a dysbiosis or disease in a patient, the method comprising identifying the bacterium using a method of identifying a bacterium for bacteriotherapy for a dysbiosis or disease as described above, and administering the identified bacterium to a patient.

The second aspect of the invention may further provide:
A method of treating a dysbiosis or disease in an individual, the method comprising identifying the bacterium using a method of identifying a bacterium for bacteriotherapy for a dysbiosis or disease as described above, and administering a therapeutically effective amount of the identified bacterium to the patient.

Many disease present with similar symptoms in the clinic and identifying the causative agent of such disease can be time-consuming and difficult, increasing costs and leading to delays for the patient until a suitable treatment can be administered. One example of such a disease is diarrheal disease which can be caused by many different microorganisms. It would therefore be advantageous if the causative agent of such diseases could be more easily identified.

The second aspect of the invention may therefore provide:
A method of diagnosing a disease in a patient, the method comprising:
obtaining a plurality of sequence reads from (e.g. by performing whole genome shotgun sequencing of DNA extracted from) a sample obtained from a patient;
using a method according to the first aspect of the invention to obtain indications of the relative abundances of lineages and/or reference genomes within the sample;
comparing the indications of the relative abundances of the lineages and/or reference genomes within the sample with the relative abundance of the lineages and/or reference genomes in a control;
identifying a bacterium with a genome, or belonging to lineage, which is present in the sample obtained from the patient but absent from the control, or which is present at a higher relative abundance in the sample obtained from the patient compared with the control;
wherein the presence, or higher relative abundance, of the bacterium in the sample is indicative of the disease.

A higher relative abundance of a bacterium may refer to a relative abundance which is 50% or more, 100% or more, 500% or more, or 1000% or more higher than the relative abundance of the bacterium in the control.

Alternatively, a higher relative abundance of a bacterium may refer to a relative abundance which is 2-fold or more, 5-fold or more, 10-fold or more, 20-fold or more, 30-fold or more, 40-fold or more, 50-fold or more, 100-fold or, 500-fold or more, or 100-fold or more higher than the relative abundance of the bacterium in the control.

The control in this context may be a sample obtained from a healthy individual, or a group of healthy individuals. Alternatively, the control may be a reference value for the expected abundance of the bacterium in a healthy individual.

A method of diagnosing a disease in a patient according to the second aspect may further comprise:
selecting the individual for treatment for the disease; or
subjecting an individual for treatment for the disease.

The treatment may be any known treatment for the disease in question.

The second aspect of the invention may provide:
A diagnostic system for use in a method according to the first aspect of the invention, the system comprising:
a tool or tools for obtaining a plurality of sequence reads from (e.g. by performing whole genome shotgun sequencing of DNA extracted from) a sample obtained from a patient; and
a computer configured (e.g. programmed) to obtain indications of the relative abundances of lineages and/or reference genomes within the sample using a method according to the first aspect from the plurality of sequence reads obtained from the sample (which may be genome shotgun sequencing data).

The second aspect of the invention may provide:
A method of treating a disease in a patient, the method comprising:
(i) requesting a test providing the results of an analysis, the test including:
obtaining a plurality of sequence reads from (e.g. by performing whole genome shotgun sequencing of DNA extracted from) a sample obtained from a patient;
using a method according to the first aspect of the invention to obtain indications of the relative abundances of lineages and/or reference genomes within the sample;
comparing the indications of the relative abundances of the lineages and/or reference genomes within the sample with the relative abundance of the lineages and/or reference genomes in a control;
identifying a bacterium with a genome, or belonging to lineage, which is present in the sample obtained from the patient but absent from the control, or which is present at a higher relative abundance in the sample obtained from the patient compared with the control;
wherein the presence, or higher relative abundance, of the bacterium in the sample is indicative of the disease;
(ii) treating the individual for the disease.

The second aspect of the invention may provide:
A method of identifying the bacterial causative agent of a disease in a patient, the method comprising:
obtaining a plurality of sequence reads from (e.g. by performing whole genome shotgun sequencing of DNA extracted from) a sample obtained from a patient;
using a method according to the first aspect of the invention, to obtain indications of the relative abundances of lineages and/or reference genomes within the sample;
comparing the indications of the relative abundances of the lineages and/or reference genomes within the sample with the relative abundance of the lineages and/or reference genomes in a control;
identifying a bacterium with a genome, or belonging to lineage, which is present in the sample obtained from the patient but absent from the control, or which is present at a higher relative abundance in the sample obtained from the patient compared with the control;
wherein said bacterium is the causative agent of the disease.

A third aspect of the invention relates to a method analysing the bacteria and/or bacteria lineages present in a sample wherein the method includes performing whole genome shotgun sequencing.

The third aspect of the invention may provide a method analysing the bacteria and/or bacterial lineages present in a sample, wherein the method includes:
performing whole genome shotgun sequencing of (i) DNA extracted from a first portion of the sample and (ii) DNA extracted from bacteria cultured from a second portion of the sample using a bacterial culturing method;
identifying one or more reference genomes and/or lineages in a database to which at least one of the plurality of sequence reads obtained in (i) is deemed to uniquely map, wherein the database stores a plurality of reference genomes and phylogenetic information which relates the stored reference genomes to each other in a phylogenetic structure;
identifying one or more reference genomes and/or lineages in the database to which at least one of the plurality of sequence reads obtained in (ii) is deemed to uniquely map;
comparing the one or more reference genomes and/or lineages to which at least one of the plurality of sequence reads obtained in (i) were deemed to uniquely map with the one or more reference genomes and/or lineages to which at least one of the plurality of sequence reads obtained in (ii) were deemed to uniquely map.

Optionally, the method according to the third aspect may comprise identifying all reference genomes and/or lineages in a database to which at least one of the plurality of sequence reads obtained in (i) is deemed to uniquely map; and
identifying all reference genomes and/or lineages in the database to which at least one of the plurality of sequence reads obtained in (ii) is deemed to uniquely map.

The method according to the third aspect may be a method of determining the bacteria and/or bacterial lineages present in a sample obtained from an individual which have or have not been cultured using a bacterial culturing method, wherein the method includes:
determining the bacteria and/or bacterial lineages present in the first portion of the sample which were and/or were not cultured using the bacterial culturing method by comparing the one or more reference genomes and/or lineages to which at least one of the plurality of sequence reads obtained in (i) were deemed to uniquely map with the one or more reference genomes and/or lineages to which at least one of the plurality of sequence reads obtained in (ii) were deemed to uniquely map.

This method may find application in, for example, determining the proportion (e.g. percentage) of bacteria and/or bacterial lineages present in a sample, such as a sample obtained from an individual which have or have not been cultured using a bacterial culturing method. This may be useful, for example, when formulating or developing a broad range medium for culturing bacteria from a sample, or comparing different broad range media with respect to proportion (e.g. percentage) of bacteria from a sample whose growth the medium can support.

A method according to the third aspect may thus be a method of determining the proportion (e.g. percentage) of bacteria present in a sample obtained from an individual which have or have not been cultured using a bacterial culturing method, wherein the method includes:
determining the proportion (e.g. percentage) of bacteria and/or bacterial lineages present in the first portion of the sample which were and/or were not cultured using the bacterial culturing method by comparing the reference genomes and/or lineages to which at least one of the plurality of sequence reads obtained in (i) were deemed to uniquely map with the reference genomes and/or lineages to which at least one of the plurality of sequence reads obtained in (ii) were deemed to uniquely map.

A method according to the third aspect may be used in the context of determining the bacteria which have been cultured using multiple, alternate, bacterial culturing methods. An alternate bacterial culturing method in this context refers to a different bacterial culturing method. The bacterial culturing methods may either be performed in parallel, using multiple portions of the same sample, or sequentially using different samples. In the latter case, the samples are preferably obtained from the same source, for example the same individual, to allow comparison between the bacteria successfully cultured using each culturing method. Alternate bacterial culturing methods may be used as part of a process for culturing the majority of the bacteria present in a sample, as different bacteria present in a sample are likely to have different growth requirements.

Where the bacterial culturing methods are employed sequentially, this may be as part of an iterative process for culturing the majority of the bacteria present in a sample, in which the bacteria and/or bacterial lineages present in a first sample which were and/or were not cultured using a first bacterial culturing method are determined, followed by determining the bacteria and/or bacterial lineages present in a second sample, preferably obtained from the same source at the first sample, e.g. the same individual, which were and/or were not cultured using a second, alternate, bacterial culturing method, and optionally determining the bacteria and/or bacterial lineages present in a second portion of the sample which were cultured using the second bacterial culturing method but which were not cultured using the first bacterial culturing method. This process can be repeated until the majority of the bacteria present in the sample have been cultured. This is useful, for example, for preparing culture collections of bacteria reflecting the majority of the bacteria present in a microbiome obtained from a healthy individual, or obtaining genomic sequences of the majority of the bacteria present in a microbiome obtained from a healthy individual and/or an individual with a dysbiosis.

A “majority” in this context may refer to culturing of at least 70%, at least 75%, at least 80%, at least 85%, at least 90%, at least 91%, at least 92%, at least 93%, at least 94%, at least 95%, at least 96%, at least 97%, at least 98%, or at least 99% of the bacterial present in a sample. “Bacteria” in this context may refer to a bacterial genus or a bacterial species. The present inventors have shown, for example, that using the broad range medium YCFA, with and without prior ethanol treatment and addition of taurocholate, bacteria from 97% of the most abundant genera and 90% of the most abundant species detected in a sample using metagenomics shotgun sequencing could be isolated. It is expected that reiteration of the culturing step using an alternate medium would allow an even higher proportion (e.g. percentage) of the bacterial genera and bacterial species present in a sample to be cultured.

A method according to the third aspect may therefore further comprise:
performing whole genome shotgun sequencing of (iii) DNA extracted from bacteria cultured from a second sample, preferably obtained from the same source, e.g. the same individual, as the sample in (i) and (ii), using an alternate bacterial culturing method;
identifying one or more reference genomes and/or lineages in the database to which at least one of the plurality of sequence reads obtained in (iii) is deemed to uniquely map;
comparing the one or more reference genomes and/or lineages to which at least one of the plurality of sequence reads obtained in (iii) were deemed to uniquely map with the one or more reference genomes and/or lineages to which at least one of the plurality of sequence reads obtained in (i) were deemed to uniquely map and, optionally, the one or more reference genomes and/or lineages to which at least one of the plurality of sequence reads obtained in (ii) were deemed to uniquely map.

Alternatively, where alternate bacterial culturing methods are used in parallel for example, a method according to the third aspect may further comprise:
performing whole genome shotgun sequencing of (iii) DNA extracted from bacteria cultured from a third portion of the sample using an alternate bacterial culturing method;
identifying one or more reference genomes and/or lineages in the database to which at least one of the plurality of sequence reads obtained in (iii) is deemed to uniquely map;
comparing the one or more reference genomes and/or lineages to which at least one of the plurality of sequence reads obtained in (iii) were deemed to uniquely map with the one or more reference genomes and/or lineages to which at least one of the plurality of sequence reads obtained in (i) were deemed to uniquely map and, optionally, the one or more reference genomes and/or lineages to which at least one of the plurality of sequence reads obtained in (ii) were deemed to uniquely map.

In this case, the method according to the third aspect may be a method of determining the bacteria and/or bacterial lineages present in a sample obtained from an individual which have been cultured using an alternate bacterial culturing method, wherein the method includes:
(a) determining the bacteria and/or bacterial lineages present in the sample which were and/or were not cultured using the alternate bacterial culturing method by comparing the one or more reference genomes and/or lineages to which at least one of the plurality of sequence reads obtained in (iii) were deemed to uniquely map with the one or more reference genomes and/or lineages to which at least one of the plurality of sequence reads obtained in (i) were deemed to uniquely map; or
(b) determining the bacteria and/or bacterial lineages present in the sample which were cultured using the alternate bacterial culturing method and which were not cultured with the bacterial culturing method by comparing the one or more reference genomes and/or lineages to which at least one of the plurality of sequence reads obtained in (iii) were deemed to uniquely map with the one or more reference genomes and/or lineages to which at least one of the plurality of sequence reads obtained in (ii) were deemed to uniquely map and the one or more reference genomes and/or lineages to which at least one of the plurality of sequence reads obtained in (i) were deemed to uniquely map.

A method according to the third aspect may be a method of preparing a culture collection of bacteria of interest present in a sample obtained from an individual, the method including identifying a bacterial culturing method for culturing the bacteria of interest, wherein the method includes:
determining the bacteria of interest present in the sample which were cultured using the bacterial culturing method by comparing the one or more reference genomes and/or lineages to which at least one of the plurality of sequence reads obtained in (i) were deemed to uniquely map with the one or more reference genomes and/or lineages to which at least one of the plurality of sequence reads obtained in (ii) were deemed to uniquely map; and
employing the bacterial culturing method to prepare a collection of cultures of said bacteria of interest from the sample.

The cultures are preferably pure cultures of bacteria. A pure culture may be a culture of a single bacterium.

In addition, a method according to the third aspect may be a method of obtaining the genomic sequence of one or more bacteria of interest present in a sample obtained from an individual, wherein the method includes:
determining the bacteria of interest present in the sample which were cultured using the bacterial culturing method by comparing the one or more reference genomes and/or lineages to which at least one of the plurality of sequence reads obtained in (i) were deemed to uniquely map with the one or more reference genomes and/or lineages to which at least one of the plurality of sequence reads obtained in (ii) were deemed to uniquely map;
employing the bacterial culturing method to prepare cultures of one or more of the bacteria of interest from the sample, and
determining the genomic sequence(s) of said bacteria.

The genomic sequence(s) of one or more bacteria of interest obtained using a method of the invention may be stored (e.g. a new reference genome) in a database that stores reference genomes (e.g. a reference database as described above). Thus, the coverage of a database that stores reference genomes (e.g. a reference database as described above) can be improved by the methods described herein. As noted below, the methods according to the first aspect may be found particularly effective when used with a database that provides thorough genome coverage in an area of interest (e.g. an area which reflects the expected content of a sample of interest).

The above second and third aspects of the present invention have been described with respect to bacteria and bacterial lineages. However, it is expected that these aspects are equally applicable to microorganisms other than bacteria, including fungi and viruses, such as bacteriophages. In this context, “microorganism” may thus refer to a bacterium, fungus, or virus. For example, it is known that microorganisms other than bacteria a present in samples obtained from humans, animals, and environmental sources, such as soil samples, as described above. DNA can be extracted from such microorganisms, or samples comprising microorganisms, and a plurality of sequence reads obtained therefrom, e.g. by performing whole genome shotgun sequencing, and analysed as described herein.

Any reference in the description of the second and third aspects of the invention to a bacterium or bacteria may thus be replaced with a reference to a microorganism or microoganisms, a fungus or fungi, or a virus or viruses (such as a bacteriophage or bacteriophages), as applicable.

Similarly, any reference to a bacterial lineage in the description of the second and third aspects of the invention may be replace with a reference to a microbial lineage, a fungal lineage or a viral lineage. For the purpose of this disclosure, a microbial lineage, which is present e.g. in a sample, can be understood as a group of microorganisms (such as a group of bacteria, fungi, or viruses) with genomes inferred as being related to each other based on one or more similarities in the genomes (e.g. using a computational technique, as is known in the art). A fungal lineage, which is present e.g. in a sample, can be understood as a group of fungi with genomes inferred as being related to each other based on one or more similarities in the genomes (e.g. using a computational technique, as is known in the art), and a viral lineage, which is present e.g. in a sample, can be understood as a group of viruses with genomes inferred as being related to each other based on one or more similarities in the genomes (e.g. using a computational technique, as is known in the art).

References to bacterial culturing methods in the description of the second and third aspects of the invention may accordingly also be replaced with references to microbial culturing methods, fungal culturing methods, or viral culturing methods, as applicable. Methods for culturing many microorganisms are known, including methods for culturing fungi and viruses.

Where the description of the second aspect of the invention refers to a method of identifying a bacterium for “bacteriotherapy” for a dysbiosis, this may be replaced with a reference to “therapy”, where the method is a method of identifying a microorganism, fungus, or virus for treatment of a dysbiosis or disease. In particular, use of bacteriophages for therapy is contemplated. Similarly, where the second aspect refers to identifying the “bacterial causative agent” of a disease, this may be replaced with “microbial causative agent”, “fungal causative agent”, or “viral causative agent”.

By way of example, the second aspect of the invention may thus provide:
A method of analysing the microorganisms and/or microbial lineages present in a sample, wherein the method includes:
obtaining a plurality of sequence reads from (e.g. by performing whole genome shotgun sequencing of DNA extracted from) (i) a first portion of a sample obtained from an individual and using a method according to the first aspect of the invention to obtain indications of the relative abundances of one or more lineages and/or reference genomes within the first portion of the sample;
obtaining a plurality of sequence reads from (e.g. by performing whole genome shotgun sequencing of DNA extracted from) (ii) microorganisms cultured from a second portion of the sample using a microbial culturing method and using a method according to the first aspect of the invention, to obtain indications of the relative abundances of one or more lineages and/or reference genomes within the cultured portion of the sample; and
comparing the indications of the relative abundances of the lineages and/or reference genomes within the first portion of the sample with the indications of the relative abundance of the lineages and/or reference genomes within the cultured sample.

Similarly, the third aspect of the invention may thus provide:
A method analysing the microorganisms and/or microbial lineages present in a sample, wherein the method includes:
performing whole genome shotgun sequencing of (i) DNA extracted from a first portion of the sample and (ii) DNA extracted from microorganisms cultured from a second portion of the sample using a microbial culturing method;
identifying one or more reference genomes and/or lineages in a database to which at least one of the plurality of sequence reads obtained in (i) is deemed to uniquely map, wherein the database stores a plurality of reference genomes and phylogenetic information which relates the stored reference genomes to each other in a phylogenetic structure;
identifying one or more reference genomes and/or lineages in the database to which at least one of the plurality of sequence reads obtained in (ii) is deemed to uniquely map;
comparing the one or more reference genomes and/or lineages to which at least one of the plurality of sequence reads obtained in (i) were deemed to uniquely map with the one or more reference genomes and/or lineages to which at least one of the plurality of sequence reads obtained in (ii) were deemed to uniquely map.

The invention also includes any combination of the aspects and preferred features described except where such a combination is clearly impermissible or expressly avoided.

The invention also includes any combination of the aspects described above with any feature or combination of features set out in relation to the database described in Annex A, except where such a combination is clearly impermissible or expressly avoided. The invention also includes any combination of the aspects described above with any feature or combination of features set out in relation to the workflow described in Annex B, except where such a combination is clearly impermissible or expressly avoided.

### BRIEF DESCRIPTION OF THE DRAWINGS

Examples of these proposals are discussed below, with reference to the accompanying drawings in which:

FIG. 1 shows an example method of using a database that stores a plurality of reference genomes and phylogenetic information which relates the stored reference genomes to each other in a phylogenetic structure.

FIG. 2(a) shows the content of a simplified example of a database that stores three reference genomes and phylogenetic information which relates the stored reference genomes to each other in a phylogenetic structure.

FIG. 2(b) shows the phylogenetic structure of the database shown in FIG. 2(a).

FIG. 3 shows the relative proportions of species reported from a sample containing equal proportions of bacterial species for kraken approach, read counts normalized to genome size (which represents the present inventors' method prior to the present invention), read counts normalized by genome uniqueness, actual percentages.

FIG. 4 shows the relative proportions of species reported from a sample containing mixed proportions of bacterial species for kraken approach, read counts normalized to genome size (which represents the present inventors' method prior to the present invention), read counts normalized by genome uniqueness, actual percentages.

FIG. 5 shows a comparison of bacteriotherapy candidates predicted to provide protection against Clostridium difficile identified through widescale analysis of co-occurrence from the Database (red) and the RePOOPULATE Study (PMCID: PMC3869191) (blue).

FIG. 6 and FIG. 7 are drawings relating to the HPMC database described in Annex A.

FIG. 8, FIG. 9 and FIG. 10 are drawings setting out an example workflow described in Annex B.

FIG. 11 shows a schematic work-flow for a process comprising identifying bacteria and/or bacterial lineages present in a faecal sample which have/have not been cultured using a set of bacterial culture conditions, adjusting the culture conditions as necessary to culture bacteria not cultured using an original set of bacterial culture conditions, obtaining the whole genome sequence and/or cultures of bacteria cultured using a suitable set of bacterial culture conditions. The steps can be performed together or separately, as applicable.

FIG. 12: Targeted phenotypic culturing facilitates bacterial discovery from healthy human faecal microbiota. FIG. 12(a) shows the relative abundance of bacteria in faecal samples (x axis) compared to relative abundance of bacterial species growing on YCFA agar plates (y axis) as determined by metagenomic sequencing. Bacteria grown on YCFA agar are representative of the complete faecal samples as indicated by R²=0.85. FIG. 12(b) shows a principal components analysis (PCoA) plot of 16S rRNA gene sequences detected from 6 donor faecal samples representing bacteria in the complete faecal samples (unfilled squares), faecal bacterial colonies recovered from YCFA agar plates without ethanol pre-treatment (filled black squares) or with ethanol pre-treatment to select for ethanol resistant spore forming bacteria (circles). Culturing without ethanol selection is representative of the complete faecal sample, ethanol treatment shifts the profile, enriching for ethanol resistant spore forming bacteria and allowing their subsequent isolation. FIG. 12(c) shows the relative abundance of bacteria grown on a culture plate after ethanol shock treatment (x axis) compared to the relative abundance of bacteria in the original faecal sample (y axis). Ethanol shock treatment of the faecal sample before culturing increased the proportion of spore forming bacteria that subsequently grew on the culture plate (as circled), allowing their isolation. Each dot represents a bacterial species.

### DETAILED DESCRIPTION

In general, the following discussion describes examples of our proposals that provide a method suitable for quantifying relative species and strain abundance from high-throughput metagenomic sequencing samples. This is achieved through specific normalization methods in the context of high quality reference genomes.

The example method shown in FIG. 1 is implemented by a database 100 and an interrogation engine 110 configured to interrogate the database 100.

The database 100 stores a plurality of reference genomes and phylogenetic information which relates the stored reference genomes to each other in a phylogenetic structure.

The interrogation engine 110 uses a plurality of sequence reads 120 obtained from a sample to count the number of sequence reads deemed to uniquely map to each of a plurality of lineages and/or reference genomes within the phylogenetic structure.

As described in more detail below, the interrogation engine 110, for each of the plurality of lineages and/or reference genomes to which at least one sequence read has been deemed uniquely mapped, normalizes the number of sequence reads counted as being deemed uniquely mapped to the lineage or reference genome using a measure that reflects the uniqueness of the lineage or reference genome so as to obtain a value indicative of the relative abundance of the lineage or reference genome within the sample, thereby obtaining indications of relative abundances 130.

In the practical example discussed below, the database 100 is the HPMC database described in more detail in Annex A.

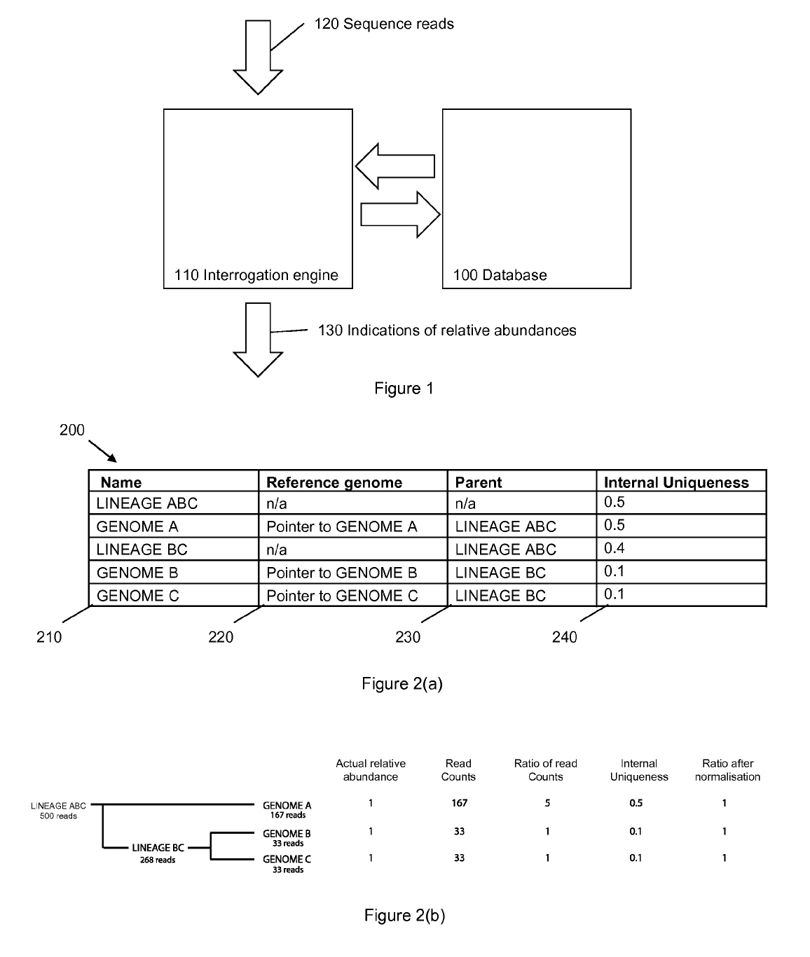

However, to provide a reader with a better understanding of the methods described herein, illustrate a method of using the database 100 in accordance with the invention, a simplified example of a database 200 is illustrated in FIG. 2(a) and the phylogenetic structure of the database 200 is illustrated in FIG. 2(b). An theoretical example showing how the database 200 can be used to provide indications of relative abundances using sequence reads obtained from a sample is provided below.

As shown in FIG. 2(a), the database 200 includes an entry for each reference genome and each lineage within the phylogenetic structure.

As shown in FIG. 2(a), the entry for each lineage/reference genome includes a name field 210 for storing a name by which the entry can be referenced.

As shown in FIG. 2(a), the entry for each reference genome further includes a reference genome field 220 for storing the reference genome or a pointer to the reference genome (this entry is blank for entries corresponding to lineages). A pointer to the reference genome is preferred since the reference genomes tend to be large in size.

As shown in FIG. 2(a), the entry for each reference genome further includes a parent field 230 for storing a pointer to a parent of the lineage/reference genome within the phylogenetic structure.

The content of the parent fields can be viewed as phylogenetic information which relates the stored reference genomes to each other in a phylogenetic structure, since an entire phylogenetic tree can be constructed from the information contained in the parent fields. Of course, phylogenetic information could be stored in numerous other ways, as would be appreciated by a skilled person.

Computational techniques for inferring such a phylogenetic structure from stored reference genomes are well known. For the HPMC database described below, the present inventors used the 16S/18S sequence to define the broad tree with closely related species resolved through whole genome alignment (preferably an on-going exercises within the database) e.g. using Mauve (PMID: 15231754), Muscle (PMID: 15034147), or MAFFT (PMID: 23329690).

As shown in FIG. 2(a), the entry for each lineage/reference genome further includes a uniqueness field 240 for storing an internal uniqueness value, which represents the proportion of the individual lineage or reference genome that is unique (relative to the genetic content of the individual lineage or reference genome).

The internal uniqueness value for each entry may be calculated by identifying one or more genetic sequences deemed to uniquely identify the corresponding lineage (if the entry is a lineage) or by identifying one or more genetic sequences deemed to uniquely identify the corresponding reference genome (if the entry is a reference genome), and then dividing the combined length of these sequences by the average length of the reference genomes in the corresponding lineage (if the entry is a lineage) or by the length of the corresponding reference genome (if the entry is a reference genome). Techniques for identifying one or more genetic sequences deemed to uniquely identify a lineage/reference genome have already been described in detail above.

Preferably, identifying one or more genetic sequences deemed to uniquely identify each lineage and reference genome in the database includes, for each reference genome in the database:
defining a plurality of segments, each segment containing a different genetic sequence from the reference genome;
for the genetic sequence contained in each segment:
   comparing the genetic sequence contained in the segment with the entirety of the genetic content of all other reference genomes in the database to establish whether the segment maps to any of the other reference genomes;
   if the genetic sequence contained in the segment maps to no other reference genome in the database, identifying the genetic sequence contained in the segment as being deemed to uniquely identify the reference genome;
   if the genetic sequence contained in the segment maps to one or more other reference genomes in the database, and if it is determined using the phylogenetic information that the genetic sequence contained in the segment maps to all of the reference genomes in a lineage and to no other reference genomes in the database, identifying the genetic sequence contained in the segment as being deemed to uniquely identify the lineage

In the present examples, the plurality of segments were obtained using a sliding window technique of length 100 base pairs and comparing the genetic sequence contained in a segment with all other reference genomes was performed with bowtie2 read aligner (see e.g. PMID: 22388286).

Referring back now to FIG. 1, the plurality of sequence reads 120 obtained from a sample are used to count the number of sequence reads deemed to uniquely map to each lineage and reference genome 100 within the phylogenetic structure stored as phylogenetic information in the database 100. Techniques for counting the number of sequence reads deemed to uniquely map to each lineage and reference genome have already been discussed above. In the present examples, the counting utilises a bowtie2 read aligner (see e.g. PMID: 22388286).

Next, for each of the plurality of lineages and/or reference genomes to which at least one sequence read has been deemed uniquely mapped, the interrogation engine 110 normalizes (by dividing) the number of sequence reads counted as being deemed uniquely mapped to the lineage or reference genome using a measure that reflects the uniqueness of the lineage or reference genome so as to obtain a value indicative of the relative abundance of the lineage or reference genome within the sample, thereby obtaining indications of relative abundances 130.

As discussed in more detail below, where all of the reference genomes stored in the database are similar or equal in length (as is assumed to be the case for the database 200 of FIG. 2(a)), the internal uniqueness value can be used as a measure that reflects the uniqueness of the corresponding lineage or reference genome.

However, as discussed in more detail below, where all of the reference genomes stored in the database are unequal in length, the internal uniqueness value is preferably adjusted (e.g. “on the fly” by the interrogation engine 110) based on the average length of the reference genomes in the corresponding lineage (if the entry is a lineage) or based on the length of the corresponding reference genome (if the entry is a reference genome) in order to provide a measure that reflects the uniqueness of the corresponding lineage or reference genome. In this case, the internal uniqueness value stored in the database can be viewed as a precursor to a measure that reflects the uniqueness of the corresponding lineage or reference genome.

Storing an internal uniqueness value in the uniqueness field 240 of the database can be useful in analyses which are not the focus of this disclosure, since this value allows a direct comparison of the percentage uniqueness between reference genomes and lineages of different lengths. Nonetheless, in other embodiments (not exemplified herein), the uniqueness field 240 of the entry for each lineage/reference genome could instead store an “global” uniqueness value that is proportional to the combined length of one or more genetic sequences deemed to uniquely identify the corresponding lineage or reference genome. In this case, the “global” uniqueness value could be used as the measure that reflects the uniqueness of the corresponding lineage or reference genome regardless of whether all of the reference genomes stored in the database are equal/unequal in length, thereby avoiding any need to adjust the internal uniqueness value “on the fly” where reference genomes stored in the database are unequal in length.

Methods described herein may be viewed as extending on the lowest common ancestor approach described in the “Background” section, above. Given thorough genome coverage provided by the HPMC database described in Annex A (which prior to the HPMC database most people would not have), a problem with applying existing approaches to uniquely classify the content of a given sample to lineages or reference genomes using the HPMC database is that very few reads could uniquely be mapped to closely related reference genomes. Adding more reference genomes to the HPMC database is helpful to identify/reduce/avoid inaccurate classification, but further reduces the number of reads that can be uniquely classified to reference genomes, especially if reference genomes share a large proportion of their genetic content (consider the extreme case of a single nucleotide polymorphism, “SNP”, between two reference genomes: only sequence reads from a sample that cover that SNP could be used to distinguish the two reference genomes in the sample).

To correct for this problem, methods described herein preferably use a measure that reflects the uniqueness of each lineage and/or reference genome, thereby taking into account the uniqueness of each lineage and/or reference genome, so as to obtain indications of the relative abundances of lineages and/or reference genomes within a sample.

Indications of relative abundances determined according to a method as described herein may be utilised in a number of different downstream applications. An example workflow in which the indications of relative abundances determined according to a method as described herein may be used is shown in Annex B.

### Theoretical Example Using the Database of FIG. 2(a)

The following theoretical example, which is provided to provide a reader with a better understanding of the methods described herein, uses the simplified database 200 of FIG. 2(a).

FIG. 2(b) provides a representation of the phylogenetic structure of the reference genomes stored in database 200.

In the example of FIG. 2(a) and FIG. 2(b), there are three reference genomes, which have internal uniqueness as follows:
GENOME A: 0.5
GENOME B: 0.1
GENOME C: 0.1

From this, and the phylogenetic information shown in FIG. 2(b), it can be inferred that GENOME A shares 50% of its genome with GENOME B and GENOME C, and GENOME B and C share 90% of their respective genomes with each other.

For simplicity, GENOME A, GENOME B and GENOME C are assumed to have the same length.

Starting with Sample A that has equal representation of each genome. Random sequence reads from Sample A should result in an equal number of sample reads being uniquely mapped to each genome.

However, due to the differing uniquenesses of the three genomes, sequence reads from Sample A will not be uniquely mapped to the three genomes in equal numbers.

For example, when you classify 1000 sequence reads from Sample A, one would expect:
˜500 to map to each of GENOME A, GENOME B or GENOME C and therefore be uniquely mapped to LINEAGE ABC (since these sequence reads can't distinguish between the 50% of genome shared between GENOME A, B and C)
˜166 to map uniquely to GENOME A
˜268 to map to each of GENOME B and GENOME C and therefore be uniquely mapped to LINEAGE BC (since these sequence reads can't distinguish between the 90% of genome shared between GENOME B and C)
˜33 to map uniquely to GENOME B
˜33 to map uniquely to GENOME C.

By counting the number of sequence reads deemed to uniquely map to each genome, the total sequence reads for each genome would be reported as:
GENOME A: ˜166 (71.5% uniqueness at genome level, 33.2% of total assigned)˜5
GENOME B: ˜33 (14.2% uniqueness at genome level, 6.6% of total assigned)˜1
GENOME C: ˜33 (14.2% uniqueness at genome level, 6.6% of total assigned)˜1

However, normalizing the counted number using internal uniqueness values provides a more accurate indication of the real relative abundances present in Sample A and allows direct comparison between all genomes and lineages in the database 200:
GENOME A: (166)/(0.5), relative abundance ˜1
GENOME B: (33)/(0.1), relative abundance ˜1
GENOME C: (33)/(0.1), relative abundance ˜1

FIG. 2(b) has been annotated with the numbers discussed above.

In the above calculations, internal uniqueness (which represents the proportion of the individual lineage or reference genome that is unique, relative to the genetic content of the individual lineage or reference genome) is used as a measure that reflects the uniqueness of the lineage or reference genome.

However, if the genomes were not equal in length, then the internal uniqueness value is preferably adjusted (e.g. “on the fly”) based on the length of the corresponding reference genome to provide a measure that reflects the uniqueness of the lineage or reference genome, which would adjust the above calculation as follows:
GENOME A: (no. reads)/(0.5×I_{A}), relative abundance ˜1
GENOME B: (no. reads)/(0.1×I_{B}), relative abundance ˜1
GENOME C: (no. reads)/(0.1×I_{C}), relative abundance ˜1

Where I_{A} is the length of GENOME A, I_{B} is the length of GENOME B, I_{C} is the length of GENOME C.

Obviously, if the database 200 were to incorporate many more reference genomes and many more sequence reads, the internal uniqueness of the reference genomes might drop. However, a fundamental benefit of the normalization approach is it allow one to adjust the read counts so that indications of relative abundances can be obtained.

Importantly, the method is not limited to obtaining relative abundances of reference genomes in a sample.

For example if for Sample A one wished to compare the relative abundance of GENOME A to LINEAGE BC, once could perform the following calculations:
GENOME A: (166)/(0.5), relative abundance ˜1
LINEAGE BC: (268)/(0.4), relative abundance ˜2

Thus, the above-exemplified method provides the ability to compare relative abundances of any genome or lineage combination through normalizing the counted numbers of sequence reads uniquely mapped to those genomes and/or lineages.

The method also works regardless of the starting composition of the sample.

For example, considering Sample B that has unequal representation of each genome in a ratio of 1:2:2 (GENOME A:GENOME B:GENOME C) the total sequence reads for each genome would be reported as:
GENOME A: ˜100 (55.6% uniqueness at genome level, 20.0% of total assigned)˜5
GENOME B: ˜40 (22.2% uniqueness at genome level, 8.0% of total assigned)˜2
GENOME C: ˜40 (22.2% uniqueness at genome level, 8.0% of total assigned)˜2

Again, normalizing the counted number using internal uniqueness values provides a more accurate indication of the real relative abundances present in Sample B:
GENOME A: (100)/(0.5), relative abundance ˜1
GENOME B: (40)/(0.1), relative abundance ˜2
GENOME C: (40)/(0.1), relative abundance ˜2

Similarly comparing GENOME A to LINEAGE BC:
GENOME A: (100)/(0.5), relative abundance ˜1
LINEAGE BC: (320)/(0.4), relative abundance ˜2

The accuracy of the genome/lineage identification and quantification is fundamentally dependent on the quality of available reference genomes in the database. As described with reference to a practical example below, the HPMC database described in Annex A, which was populated with reference genomes using techniques described in this application, can be used to provide useful results in the case of gut flora. Without access to a database storing a comprehensive collection of reference genomes relevant to a sample under study, results may be less useful.

Assuming the database provides a comprehensive collection of reference genomes, the resolution of classification may be limited by sequencing depth. Accordingly, the number of sequence of sequence reads to be obtained may be chosen to be at least m/u, where m is a minimum number of reads deemed appropriate for confident detection of a lineage or reference genome of interest, and where u is the internal uniqueness, which represents the proportion of the individual lineage or reference genome that is unique (relative to the genetic content of the individual lineage or reference genome). For a typical experiment, m is preferably 100 or more, more preferably 1000 or more.

A skilled person would appreciate that whilst internal uniqueness has been used to normalize counts in the above example, the specific form of the measure used to normalize counted numbers of sequence reads is not important, so long as it reflects the uniqueness of the lineages and/or reference genomes being studied.

### Practical Example Using the HPMC Database Described in Annex A—Controlled Data

To demonstrate the practical effectiveness of the methods described herein, it is possible to considered 5 species Aspergillus fumigatus, Bifidobacterium breve 689b, Bifidobacterium breve S27, Clostridium difficile 630 and Staphylococcus phage K. This selection simultaneously demonstrates the method is effective on eukaryotic components of the microbiota (Aspergillus) with large genomes (29.3 Mb) and bacteriophage (Staphylococcus phage K, 0.01 Mb genome). It also demonstrates the ability to differentiate the two strains of B. breve (genome size ˜2.3 Mb) and a distantly related bacteria C. difficile.

To demonstrate the effectiveness of this method it is necessary to utilize real sequencing reads to capture variability observed in real sequence reads. However, in this case it is not possible to know the “true” metagenomic content of a metagenomic sample. To overcome this limitation sequencing reads obtained from direct genome sequencing are sampled at a prescribed percentage to generate pseudo-metagenomic sequencing reads at known proportions.

The measure used to normalize counts is essential to the method, but the specific form of the measure and the detail with which it is calculated is not important for the method's success, so long as it reflects the uniqueness of the lineages and/or reference genomes being studied.

For this example, the uniqueness measure used to normalize counts was calculated by using a 100 bp sliding window approach. The genome and lineage uniqueness used to normalize counts was reported as the percentage of 100 bp regions that would uniquely identify the genome or lineage against all other genomes within the database. The comparison was performed using the bowtie2 algorithm with standard parameters. Read abundance levels were then weighted by this measure as described above to determine the relative species abundance from the relative read abundance.

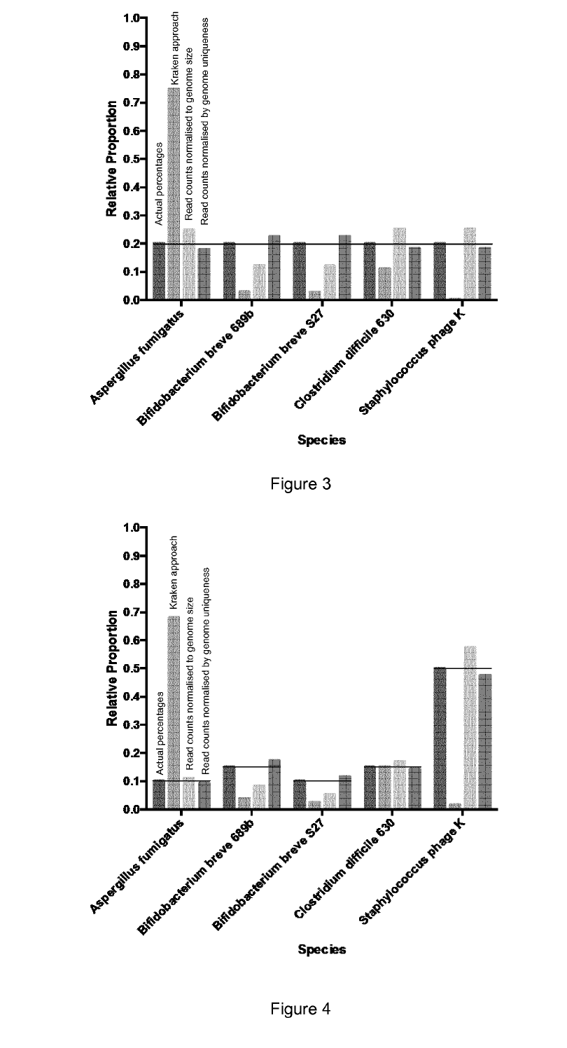

Sample containing equal proportions (FIG. 3):
1:1:1:1:1 Aspergillus:B. breve 689b:B. breve S27:C. difficile 630:Saphylococcus phage K

Sample containing mixed proportions (FIG. 4):
2:3:2:3:10 Aspergillus:B. breve 689b:B. breve S27:C. difficile 630:Saphylococcus phage K

Sequence reads were randomly selected from the complete genome sequences of each species and assembled into a pseudo-metagenomic sample with known read proportions. Read abundance levels are then weighted by this “uniqueness factor” as described above to determine the relative species abundance from the relative read abundance.

Applying the uniqueness normalization to the sample containing equal proportions:
Aspergillus: ˜1
B. breve 689b: ˜1
B. breve S27: ˜1
C. difficile 630: ˜1
Saphylococcus phage K: ˜1

Applying the uniqueness normalization to the sample containing mixed proportions:
Aspergillus: ˜2
B. breve 689b: ˜3
B. breve S27: ˜2
C. difficile 630: ˜3
Saphylococcus phage K: ˜10

It is also possible to calculate the relative abundances of any particular lineage using this method. In this example there are two strains of B. breve represented. Considering these two strains as a single B. breve lineage, uniqueness normalization for the sample containing equal proportions provides results as follows:
Aspergillus: ˜1
B. breve Lineage: ˜2
C. difficile 630: ˜1
Saphylococcus phage K: ˜1

Using uniqueness normalization for the sample containing equal proportions provides results as follows:
Aspergillus: ˜2
B. breve Lineage: ˜5
C. difficile 630: ˜3
Saphylococcus phage K: ˜10

Note that calculating relative abundance for a lineage involves counting the number of sequence reads deemed to uniquely map to the lineage and normalizing that count using a measure that reflects the uniqueness of the lineage, rather than just adding the relative abundances determined for individual members of the lineage (though the result should come out as similar—as above—assuming that there is good coverage of the lineage in the database).

Practical Example Using the HPMC Database Described in Annex A—Real data

To demonstrate the practical benefits of this approach it is possible to consider many examples where identification of specific species or strains can provide important insights to biology or bacteriotherapy candidate design as it provide exact species or strains as opposed to genera or family level approximations.

### Example 1: Bacteriotherapy Candidate Identification

One specific example is the identification of C. difficile bacteriotherapy candidates. When applying this analysis approach to 435 public metagenomic samples where C. difficile is detected in individuals that report normal health it is possible to also identify commonly co-occurring species that are likely to play a role in maintaining health and preventing uncontrolled C. difficile expansion (and thus disease). This analysis identifies 30 species that commonly associate with asymptomatic C. difficile carriers (p<0.01). When compared to the publicly available RePOOPULATE study (PMC3869191) 24 of the 25 species identified were represented in this list (Eubacterium desmolans was absent).

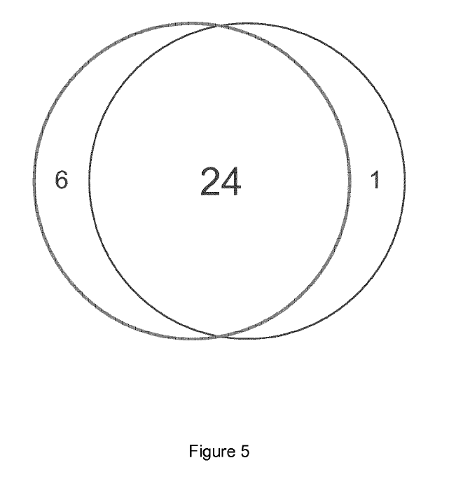

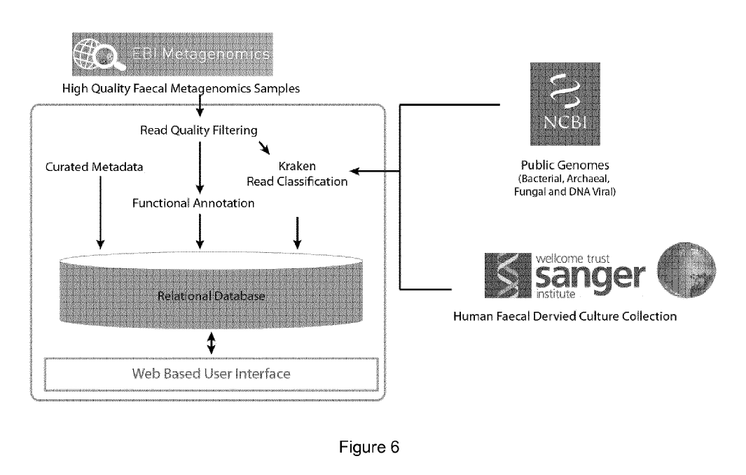

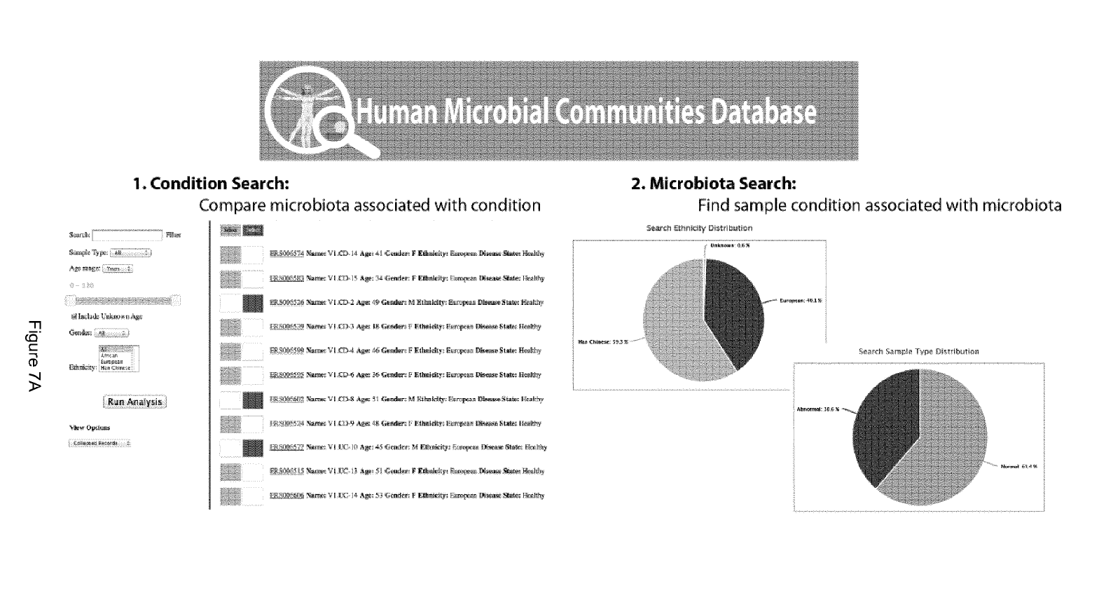

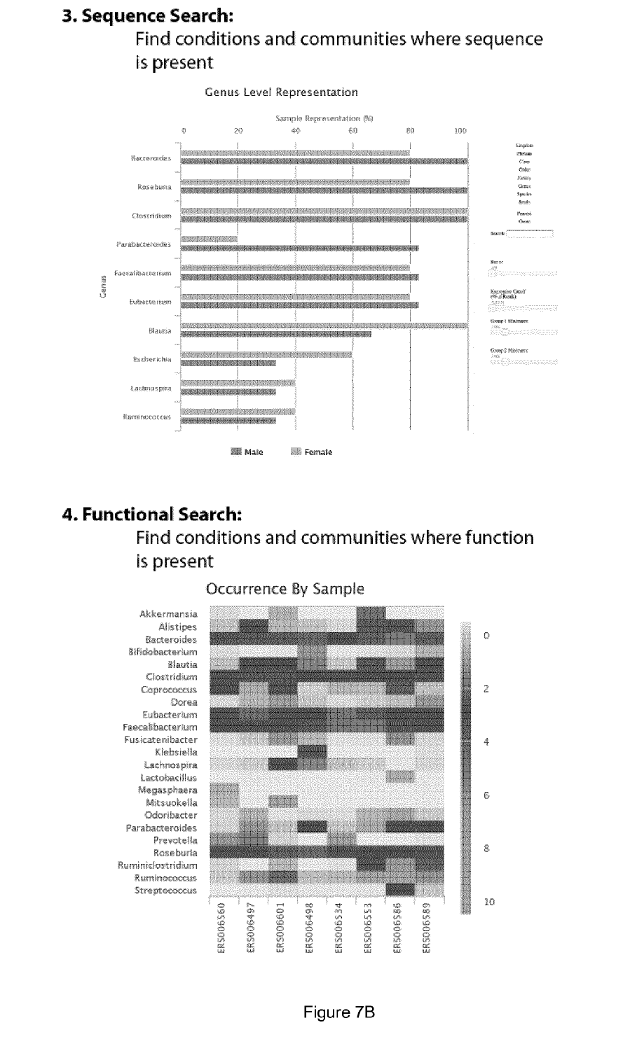

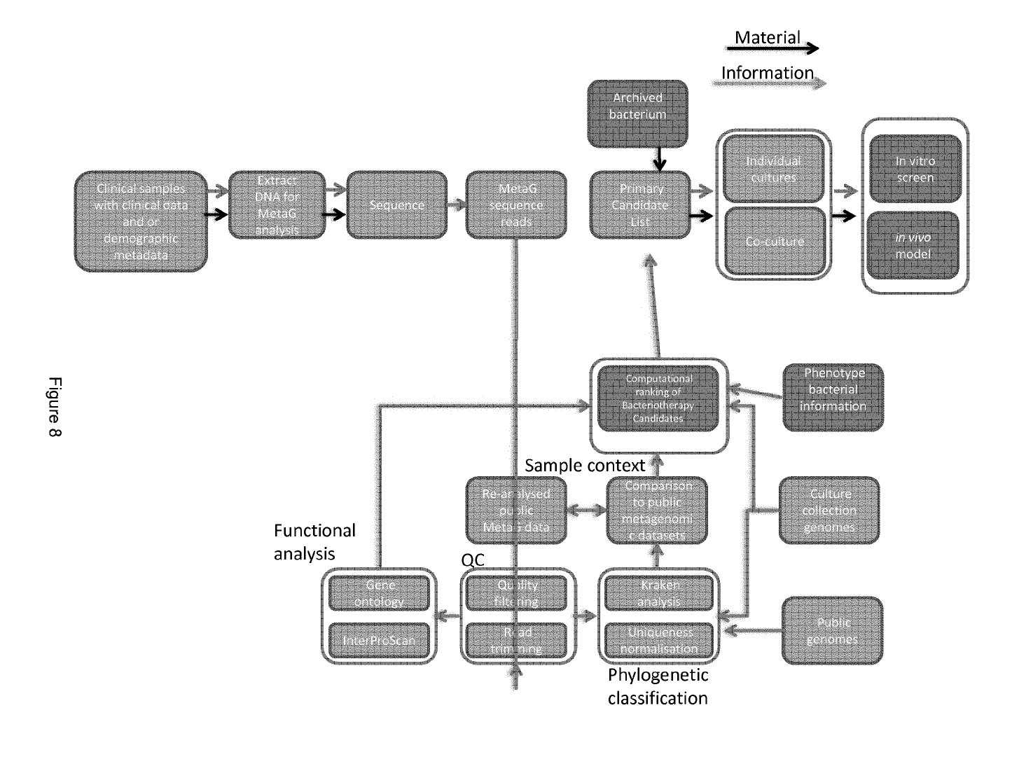

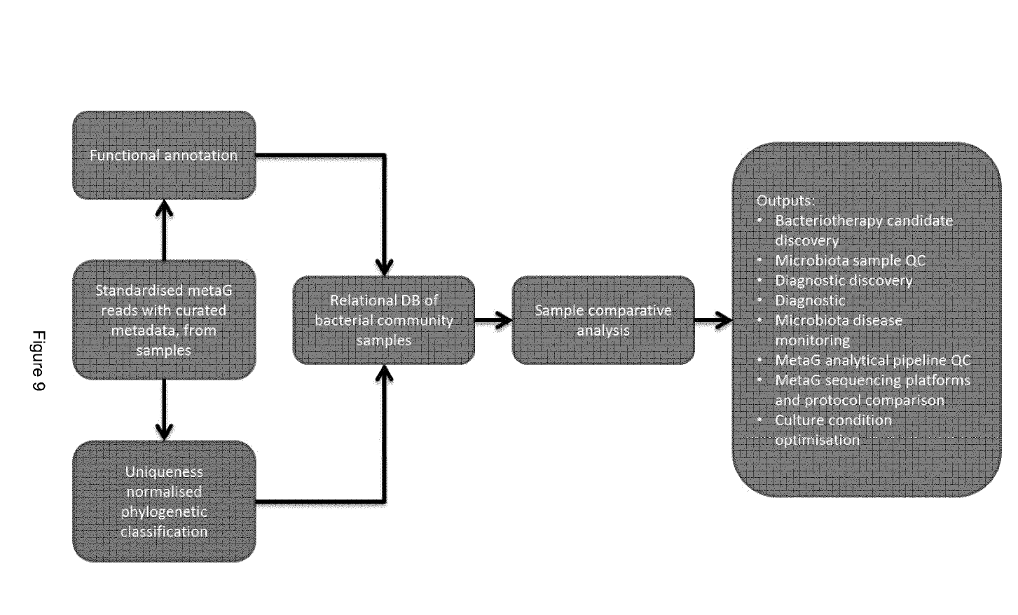

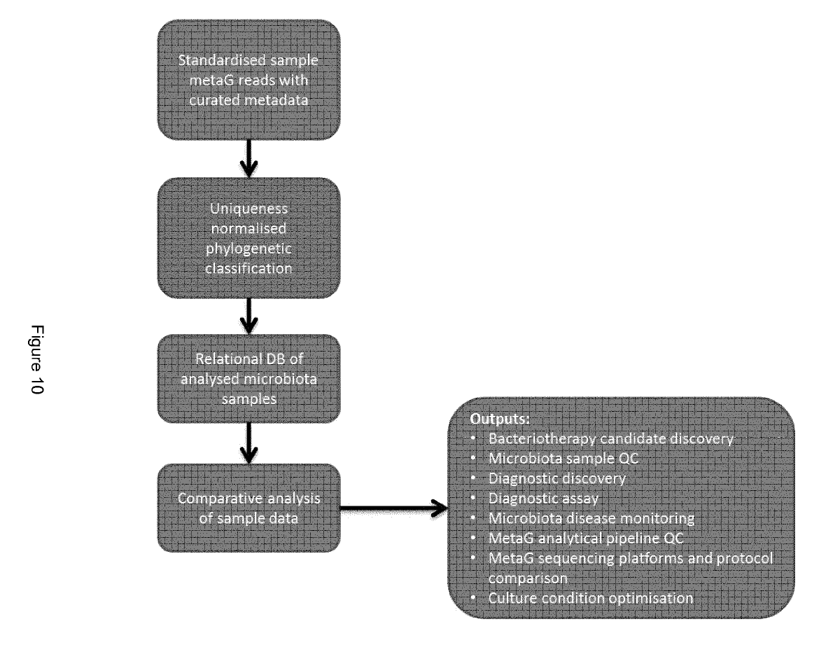

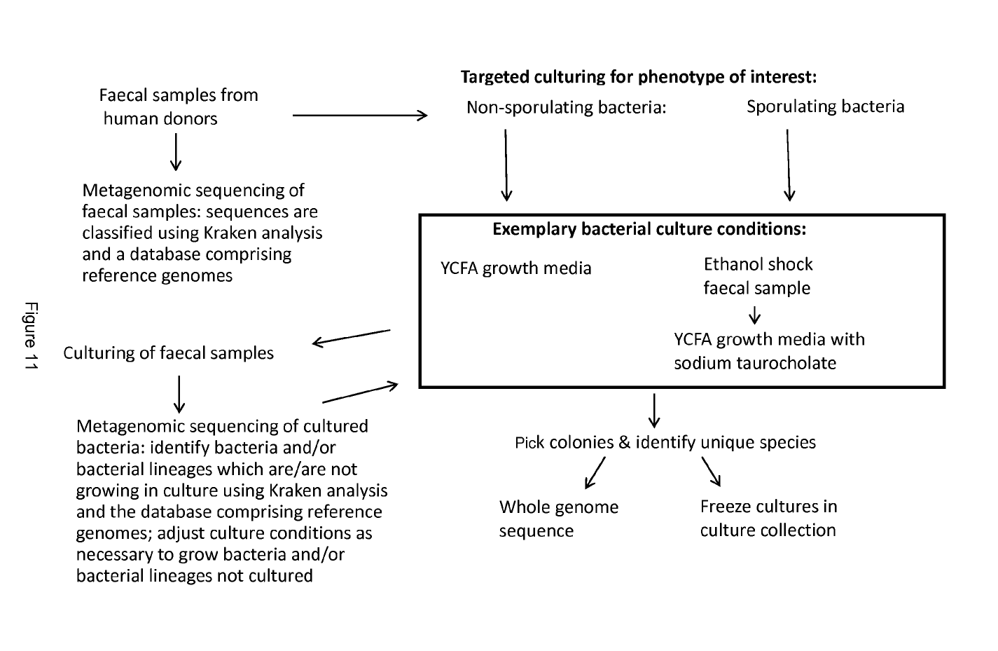

FIG. 5 shows a comparison of bacteriotherapy candidates predicted to provide protection against Clostridium difficile identified through widescale analysis of co-occurrence from the Database (left) and the RePOOPULATE Study (PMCID: PMC3869191) (right).

### Example 2: Biomarker Identification

Accurate, species and strain level pathogen and commensal identification will provide an important tool for metagenomic based diagnostics and biomarker identification. The proposed method could be utilized to identify the specific strains of a particular pathogen, such as identification of epidemic (027 ribotype) in a C. difficile infected individual. This approach has many applications in clinical setting where the rapid, accurate, pathogen identification is of critical importance. Such an approach could also be critical in the identification of biomarkers suitable for identification or stratification of those at risk to microbiota mediated disease.

Described below are examples of methods of the invention for identifying bacteria and/or bacterial lineages present in a sample, such as bacteria and/or bacterial lineages present in a sample which have/have not been cultured using a set of bacterial culture conditions, adjusting culture conditions as necessary to culture bacteria not cultured using an original set of bacterial culture conditions, obtaining whole genomic sequences and/or cultures of bacteria cultured using a suitable set of bacterial culture conditions.

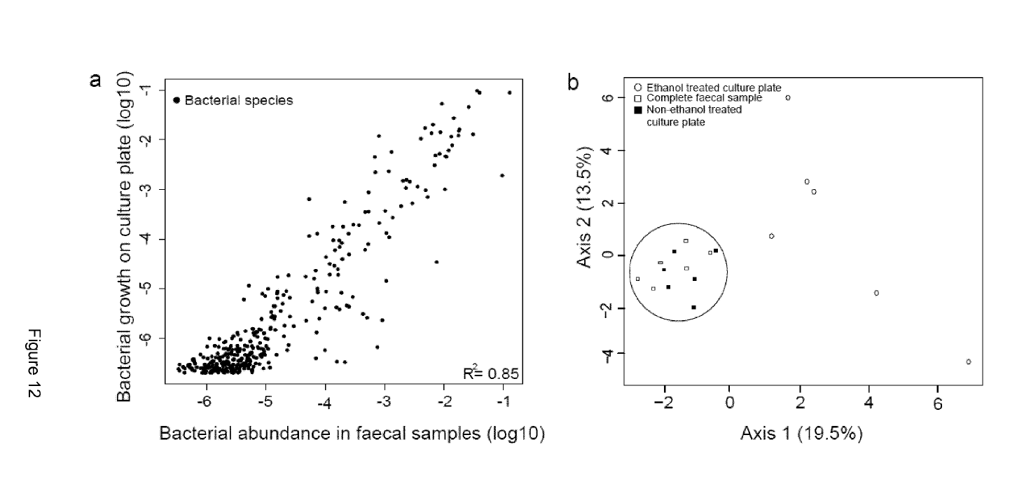

A schematic diagram of a work-flow encompassing the above methods is shown in FIG. 11. The methods encompassed by the depicted work-flow can be performed separately, where applicable. For example, a method of identifying bacteria and/or bacterial lineages present in a sample as shown schematically on the left side of FIG. 11 may be performed with or without performing an iteration of the culturing and metagenomics sequencing steps using alternate culture conditions.

### Example 3—Analysis of the Proportion of Bacteria Present in a Sample which Has been Cultured

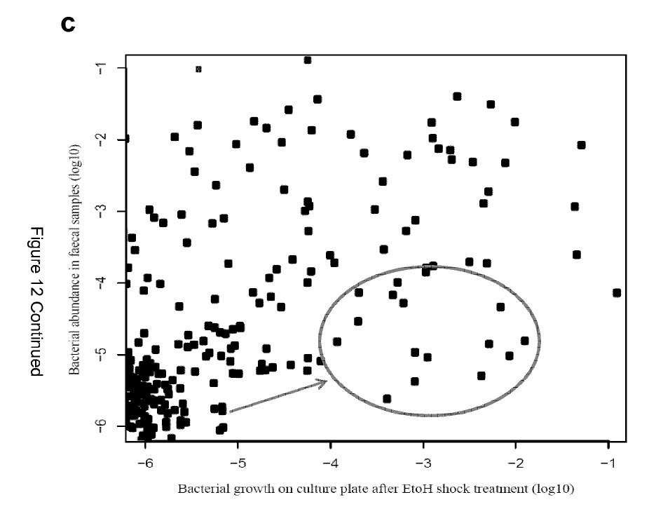

Faecal samples from 6 healthy humans were collected and the resident bacterial communities defined using a combined metagenomic sequencing and bacterial culturing approach using the complex, broad range culture medium, YCFA (Duncan et al., 2002). Applying shotgun metagenomic sequencing we profiled and compared the bacterial species present in the original faecal samples to those that grew on YCFA agar plates (by scraping the colonies off the plate for DNA isolation and sequencing). Importantly, we observed a strong correlation between the two (R²=0.85) (FIG. 12A) demonstrating that a significant proportion of the bacteria within the faecal microbiota can be cultured with a single growth medium. However, more than 8×10⁶ colonies would need to be picked and screened from agar plates to match the species detection sensitivity of metagenomic sequencing. Thus, we established a broad range culturing method that can be used to strategically identify novel bacteria with a defined phenotype.

The human intestinal microbiota is dominated by strict anaerobic bacteria that are extremely sensitive to ambient oxygen, so it is not known how these bacteria survive environmental exposure to transmit between individuals. Certain pathogenic Firmicutes, such as the diarrheal pathogen Clostridium difficile, produce metabolically dormant and highly resistant spores during colonization that facilitate both persistence within the host and environmental survival once shed (Francis et al., 2013; Janoir et al., 2013; Lawley et al., 2009). C. difficile spores have evolved mechanisms to resume metabolism and vegetative growth after intestinal colonisation by germinating in response to digestive bile acids (Francis et al., 2013). Relatively few intestinal spore-forming bacteria have been cultured to date and their genomes, phylogenies and phenotypes remain poorly characterised (Rajilic-Stojanovic et al., 2014). Recently, metagenomic studies have suggested that other unexpected members of the intestinal microbiota possess potential sporulation genes, even though these bacteria have never been grown in a laboratory and are not known to produce spores (Galperin et al., 2012; Abecasis et al., 2013; Meehan et al., 2014).

We hypothesized that sporulation is an unappreciated basic phenotype of the human intestinal microbiota that may have a profound impact on microbiota persistence and spread between humans. Spores from C. difficile are resistant to ethanol and this phenotype can be used to select for spores from a mixed population of spores and sensitive vegetative cells (Riley et al., 1987). Faecal samples were treated with ethanol and analysed using our combined culture and metagenomic approach. Principle component analysis demonstrated that ethanol treatment profoundly altered the culturable bacterial composition compared to the original profile and efficiently enriched for ethanol resistant bacteria, facilitating their isolation (FIG. 12B). We next isolated individual bacterial colonies from faecal samples that grew on the YCFA medium with or without prior ethanol treatment and assigned them to a species or classified them as novel. We picked ˜2000 bacterial colonies from both ethanol treated and ethanol non-treated conditions, re-streaked them to purity and performed full-length 16S rRNA gene sequencing. Sodium taurocholate can be added to the growth media to stimulate spore germination. The 16S rRNA gene sequences were compared to The Ribosomal Database Project (RDP) reference database to assign genus level taxonomy (Wang et al., 2007). We imposed a BLASTn identity threshold of ≥98.7% over the full-length 16S rRNA gene sequence to define either a characterised or novel species (Clarridge et al., 2004; Bosshard et al., 2003; Altschul et al., 1990), and these were archived as frozen stocks for future analysis.

In total, we isolated bacteria from 97% of the most abundant genera and 90% of the most abundant species detected in our cohort by metagenomic sequencing. Even bacterial genera that were present at low relative abundance (<0.1%) in the faecal samples were cultured. Overall, we cultured and archived 137 distinct bacterial species which included 45 novel species, and isolates representing 20 novel genera and 2 novel families. Our collection contains 90 species from the Human Microbiome Project's ‘most wanted’ list of previously uncultured and unsequenced microbes (Fodor et al., 2012). Thus, our broad-range culture approach led to massive bacterial discovery and challenges the notion that the majority of the intestinal microbiota is “unculturable”.

By obtaining and then storing the genomic sequences of the bacterial isolates in a database, a database having more thorough genome coverage of intestinal microbiota, such as the HPMC database described in Annex A can be established.

### FINAL REMARKS

When used in this specification and claims, the terms “comprises” and “comprising”, “including” and variations thereof mean that the specified features, steps or integers are included. The terms are not to be interpreted to exclude the possibility of other features, steps or integers being present.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

### REFERENCES

All references referred to herein are hereby incorporated by reference.

Abecasis, A. B. et al. A genomic signature and the identification of new sporulation genes. Journal of bacteriology 195, 2101-2115, doi:10.1128/JB.02110-12 (2013).

Altschul, S. F., Gish, W., Miller, W., Myers, E. W. & Lipman, D. J. Basic local alignment search tool. Journal of molecular biology 215, 403-410, doi:10.1016/S0022-2836(05)80360-2 (1990).

Bosshard, P. P., Abels, S., Zbinden, R., Bottger, E. C. & Altwegg, M. Ribosomal DNA sequencing for identification of aerobic gram-positive rods in the clinical laboratory (an 18-month evaluation). J Clin Microbiol 41, 4134-4140 (2003).

Clarridge, J. E., 3rd. Impact of 16S rRNA gene sequence analysis for identification of bacteria on clinical microbiology and infectious diseases. Clinical microbiology reviews 17, 840-862, table of contents, doi:10.1128/CMR.17.4.840-862.2004 (2004).

Duncan, S. H., Hold, G. L., Harmsen, H. J., Stewart, C. S. & Flint, H. J. Growth requirements and fermentation products of Fusibacterium prausnitzii, and a proposal to reclassify it as Faecalibacterium prausnitzii gen. nov., comb. nov. Int J Syst Evol Microbiol 52, 2141-2146 (2002).

Fodor, A. A. et al. The “most wanted” taxa from the human microbiome for whole genome sequencing. PloS one 7, e41294, doi:10.1371/journal.pone.0041294 (2012).

Francis, M. B., Allen, C. A., Shrestha, R. & Sorg, J. A. Bile acid recognition by the Clostridium difficile germinant receptor, CspC, is important for establishing infection. PLoS pathogens 9, e1003356, doi:10.1371/journal.ppat.1003356 (2013).

Galperin, M. Y. et al. Genomic determinants of sporulation in Bacilli and Clostridia: towards the minimal set of sporulation-specific genes. Environmental microbiology 14, 2870-2890, doi:10.1111/j.1462-2920.2012.02841.x (2012).

Goodman et al., PNAS, vol. 108, 6252-6257 (2011)

Janoir, C. et al. Adaptive strategies and pathogenesis of Clostridium difficile from in vivo transcriptomics. Infect Immun 81, 3757-3769, doi:10.1128/IAI.00515-13 (2013).

Lawley, T. D. and A. W. Walker (2013). “Intestinal colonization resistance.” Immunology 138(1): 1-11.

Lawley, T. D. et al. Antibiotic treatment of clostridium difficile carrier mice triggers a supershedder state, spore-mediated transmission, and severe disease in immunocompromised hosts. Infect Immun 77, 3661-3669, doi:10.1128/IAI.00558-09 (2009).

Meehan, C. J. & Beiko, R. G. A phylogenomic view of ecological specialization in the Lachnospiraceae, a family of digestive tract-associated bacteria. Genome biology and evolution 6, 703-713, doi:10.1093/gbe/evu050 (2014).

Petrof, E. O., G. B. Gloor, S. J. Vanner, S. J. Weese, D. Carter, M. C. Daigneault, E. M. Brown, K. Schroeter and E. Allen-Vercoe (2013). “Stool substitute transplant therapy for the eradication of Clostridium difficile infection: ‘RePOOPulating’ the gut.” Microbiome 1(1): 3.

Rajilic-Stojanovic, M. & de Vos, W. M. The first 1000 cultured species of the human gastrointestinal microbiota. FEMS microbiology reviews 38, 996-1047, doi:10.1111/1574-6976.12075 (2014).

Riley, T. V., Brazier, J. S., Hassan, H., Williams, K. & Phillips, K. D. Comparison of alcohol shock enrichment and selective enrichment for the isolation of Clostridium difficile. Epidemiology and infection 99, 355-359 (1987).

Seekatz, A. M., J. Aas, C. E. Gessert, T. A. Rubin, D. M. Saman, J. S. Bakken and V. B. Young (2014). “Recovery of the gut microbiome following fecal microbiota transplantation.” MBio 5(3): e00893-00814.

Stewart, E. J. (2012). “Growing unculturable bacteria.” J Bacteriol 194(16): 4151-4160.

van Nood, E., A. Vrieze, M. Nieuwdorp, S. Fuentes, E. G. Zoetendal, W. M. de Vos, C. E. Visser, E. J. Kuijper, J. F. Bartelsman, J. G. Tijssen, P. Speelman, M. G. Dijkgraaf and J. J. Keller (2013). “Duodenal infusion of donor feces for recurrent Clostridium difficile.” N Engl J Med 368(5): 407-415.

Wang, Q., Garrity, G. M., Tiedje, J. M. & Cole, J. R. Naive Bayesian classifier for rapid assignment of rRNA sequences into the new bacterial taxonomy. Applied and environmental microbiology 73, 5261-5267, doi:10.1128/AEM.00062-07 (2007).

1. A method of using a database that stores a plurality of reference genomes and phylogenetic information which relates the stored reference genomes to each other in a phylogenetic structure, the method including:
using a plurality of sequence reads obtained from a sample to count the number of sequence reads deemed to uniquely map to each of a plurality of lineages and/or reference genomes within the phylogenetic structure;
for each of the plurality of lineages and/or reference genomes to which at least one sequence read has been deemed uniquely mapped, normalizing the number of sequence reads counted as being deemed uniquely mapped to the lineage or reference genome using a measure that reflects the uniqueness of the lineage or reference genome so as to obtain an indication of the relative abundance of the lineage or reference genome within the sample.

2. A method according to claim 1, wherein the method includes, for each of the plurality of lineages and/or reference genomes, determining a measure that reflects the uniqueness of the lineage or reference genome or a precursor of such a measure by:
for the/each reference genome:
identifying one or more genetic sequences deemed to uniquely identify the reference genome;
determining a measure that reflects the uniqueness of the reference genome or a precursor of such a measure based on the one or more genetic sequences deemed to uniquely identify the reference genome;
for the/each lineage:
identifying one or more genetic sequences deemed to uniquely identify the lineage;
determining a measure that reflects the uniqueness of the lineage or a precursor of such a measure based on the one or more genetic sequences deemed to uniquely identify the lineage.

3. A method according to claim 2, wherein identifying one or more genetic sequences deemed to uniquely identify each of the plurality of lineages and/or reference genomes includes, for each reference genome in the database:
defining a plurality of segments, each segment containing a different genetic sequence from the reference genome;
for the genetic sequence contained in each segment:
comparing the genetic sequence contained in the segment with the majority of the genetic content of all other reference genomes in the database to establish whether the segment maps to any of the other reference genomes;
if the genetic sequence contained in the segment maps to no other reference genome in the database, identifying the genetic sequence contained in the segment as being deemed to uniquely identify the reference genome;
if the genetic sequence contained in the segment maps to one or more other reference genomes in the database, and if it is determined using the phylogenetic information that the genetic sequence contained in the segment maps to at least a majority of the reference genomes in a lineage, identifying the genetic sequence contained in the segment as being deemed to uniquely identify the lineage.

4. A method according to claim 2, wherein identifying one or more genetic sequences deemed to uniquely identify each of the plurality of lineages and/or reference genomes includes, for each reference genome in the database:
defining a plurality of segments, each segment containing a different genetic sequence from the reference genome;
for the genetic sequence contained in each segment:
comparing the genetic sequence contained in the segment with the entirety of the genetic content of all other reference genomes in the database to establish whether the segment maps to any of the other reference genomes;
if the genetic sequence contained in the segment maps to no other reference genome in the database, identifying the genetic sequence contained in the segment as being deemed to uniquely identify the reference genome;
if the genetic sequence contained in the segment maps to one or more other reference genomes in the database, and if it is determined using the phylogenetic information that the genetic sequence contained in the segment maps to all of the reference genomes in a lineage and to no other reference genomes in the database, identifying the genetic sequence contained in the segment as being deemed to uniquely identify the lineage.

5. A method according to any one of claims 2 to 4, wherein identifying one or more genetic sequences deemed to uniquely identify each of the plurality of lineages and/or reference genomes is performed before the sequence reads are obtained from the sample.

6. A method according to any one of claims 3 to 5, wherein the plurality of segments defined for each reference genome have a predetermined length, and include each possible segment of that length that could be defined for the reference genome.

7. A method according to any previous claim, wherein using a plurality of sequence reads obtained from a sample to count the number of sequence reads deemed to uniquely map to each of a plurality of lineages and/or reference genomes within the phylogenetic structure includes:
for the/each reference genome:
comparing the plurality of sequence reads with the reference genome to establish whether any sequence reads map to the reference genome and to no other reference genome stored in the database;
if a sequence read maps to the reference genome and to no other reference genome stored in the database, counting the sequence read as being deemed to uniquely map to the lineage;
for the/each lineage:
comparing the plurality of sequence reads with one or more genetic sequences deemed to uniquely identify the lineage to establish whether any sequence reads map to any of the identified one or more genetic sequences;
if a sequence read maps to any of the one or more genetic sequences deemed to uniquely identify the lineage, counting the sequence read as being deemed to uniquely map to the lineage.

8. A method according to any previous claim, wherein the database includes an entry for each reference genome and each lineage within the phylogenetic structure.

9. A method according to claim 8, wherein, the entry for each lineage/reference genome includes a parent field for storing a pointer to a parent of the lineage/reference genome within the phylogenetic structure.

10. A method according to claim 8 or 9, wherein the entry for each lineage/reference genome includes a uniqueness field for storing a measure that reflects the uniqueness of the lineage or reference genome or a precursor of such a measure.

11. A method according to claim 10, wherein the uniqueness field is recalculated each time a new reference genome is stored in the database.

12. A method according to any previous claim, wherein the method includes obtaining the plurality of sequence reads from the sample

13. A method according to claim 12, wherein the sequence reads are obtained by a shotgun sequencing process in which the DNA contained in the sample is broken up randomly into small segments which are then sequenced to obtain the plurality of sequence reads, wherein the plurality of sequence reads from the sample are obtained from across the complete DNA of organisms within the sample.

14. A method according to any previous claim, wherein the sequence reads each have a length of at least 80 or more base pairs.

15. An apparatus including a computer configured to perform a method according to any previous claim.

16. A computer-readable medium having computer-executable instructions configured to cause a computer to perform a method according to any of claims 1 to 14.

17. A method of analysing the bacteria and/or bacterial lineages present in a sample, wherein the method includes:
obtaining a plurality of sequence reads from (i) a first portion of a sample and using a method according to any one of claims 1 to 14 to obtain indications of the relative abundances of one or more lineages and/or reference genomes within the first portion of the sample;
obtaining a plurality of sequence reads from (ii) bacteria cultured from a second portion of the sample using a bacterial culturing method and using a method according to any one of claims 1 to 14, to obtain indications of the relative abundances of one or more lineages and/or reference genomes within the cultured portion of the sample; and
comparing the indications of the relative abundances of the lineages and/or reference genomes within the first portion of the sample with the indications of the relative abundance of the lineages and/or reference genomes within the cultured sample.

18. A method according to claim 17, wherein the method is a method of determining the bacteria and/or bacterial lineages present in a sample which have or have not been cultured using a bacterial culturing method, wherein the method includes:
determining the bacteria and/or bacterial lineages present in the first portion of the sample which were and/or were not cultured using the bacterial culturing method by comparing the indications of the relative abundances of the lineages and/or reference genomes within the first portion of sample with the indications of the relative abundance of the lineages and/or reference genomes within the cultured sample.

19. A method according to claim 17 or 18, further comprising:
obtaining a plurality of sequence reads from (iii) bacteria cultured from a second sample, obtained from the same source as the sample in (i) and (ii), using an alternate bacterial culturing method and using a method according to any one of claims 1 to 14, to obtain indications of the relative abundances one or more lineages and/or reference genomes within the alternately cultured sample; and
comparing the indications of the relative abundances of the lineages and/or reference genomes within the alternately cultured sample with the indications of the relative abundance of the lineages and/or reference genomes within the first portion of the sample and, optionally, the indications of the relative abundance of the lineages and/or reference genomes within the cultured sample.

20. A method according to claim 19, wherein the method is a method of determining the bacteria and/or bacterial lineages present in a sample which have been cultured using an alternate bacterial culturing method, wherein the method includes:
(a) determining the bacteria and/or bacterial lineages present in the sample which were and/or were not cultured using the alternate bacterial culturing method by comparing the indications of the relative abundances of the lineages and/or reference genomes within the alternately cultured sample with the indications of the relative abundance of the lineages and/or reference genomes within the first portion of the sample; or
(b) determining the bacteria and/or bacterial lineages present in the sample which were cultured using the alternate bacterial culturing method and which were not cultured with the bacterial culturing method by comparing the indications of the relative abundances of the lineages and/or reference genomes within the alternately cultured sample with the indications of the relative abundance of the lineages and/or reference genomes within the first portion of the sample and the relative abundance of the lineages and/or reference genomes within cultured sample.

21. A method according to claim 17, wherein the method is a method of preparing a culture collection of bacteria of interest present in a sample, the method including identifying a bacterial culturing method for culturing the bacteria of interest, wherein the method includes:
determining the bacteria of interest present in the sample which were cultured using the bacterial culturing method by comparing the indications of the relative abundances of the lineages and/or reference genomes within the first portion of sample with the indications of the relative abundance of the lineages and/or reference genomes within the cultured sample; and
employing the bacterial culturing method to prepare a collection of cultures of said bacteria of interest from the sample.

22. A method according to claim 17, wherein the method is a method of obtaining the genomic sequence of one or more bacteria of interest present in a sample, wherein the method includes:
determining the bacteria of interest present in the sample which were cultured using the bacterial culturing method by comparing the indications of the relative abundances of the lineages and/or reference genomes within the first portion of sample with the indications of the relative abundance of the lineages and/or reference genomes within the cultured sample;
employing the bacterial culturing method to prepare cultures of one or more of the bacteria of interest from the sample, and
determining the genomic sequence(s) of said bacteria.

23. A method according to claim 22, further comprising adding the genomic sequence(s) of said one or more of the bacteria of interest to a database that stores reference genomes.

24. A method of identifying a bacterium for bacteriotherapy for a dysbiosis, the method comprising:
obtaining a plurality of sequence reads from a sample obtained from a patient with the dysbiosis;
using a method according to any one of claims 1 to 14 to obtain indications of the relative abundances of lineages and/or reference genomes within the sample;
comparing the indications of the relative abundances of the lineages and/or reference genomes within the sample with the relative abundance of the lineages and/or reference genomes in a control;
selecting a bacterium with a genome, or belonging to a lineage, which is absent from the sample obtained from the patient but present in the control, or which is present at a lower relative abundance in the sample obtained from the patient compared with the control, for bacteriotherapy for the dysbiosis.

25. A method of identifying a bacterium for bacteriotherapy for a dysbiosis, the method comprising:
obtaining a plurality of sequence reads from (i) a first sample obtained from a patient with the dysbiosis;
obtaining a plurality of sequence reads from (ii) a second sample obtained from the same patient after the patient has received a faecal transplant;
using a method according to any one of claims 1 to 14 to obtain indications of the relative abundances of lineages and/or reference genomes within the sample; comparing the indications of the relative abundances of the lineages and/or reference genomes within the first sample with the relative abundance of the lineages and/or reference genomes in the second sample;
selecting a bacterium with a genome, or belonging to lineage, which is absent from the first sample but present in the second sample, or which is present at a lower relative abundance in the first sample compared with the second sample, for bacteriotherapy for the dysbiosis.

26. A method of identifying a bacterium for therapy of a disease characterised by the presence of a pathogenic bacterium, the method comprising:
obtaining a plurality of sequence reads from (i) a first sample obtained from a first asymptomatic carrier of the pathogenic bacterium and obtaining a plurality of sequence reads from (ii) a second sample obtained from a second asymptomatic carrier of the pathogenic bacterium;
using a method according to any one of claims 1 to 14 to obtain indications of the relative abundances of lineages and/or reference genomes within the first sample and the second sample;
comparing the indications of the relative abundances of the lineages and/or reference genomes within the first sample with the relative abundance of the lineages and/or reference genomes in the second sample;
selecting a bacterium with a genome, or belonging to a lineage, which is common to the first and second sample for bacteriotherapy for the disease.

27. A method of identifying a bacterium for therapy of a disease characterised by the presence of a pathogenic bacterium, the method comprising:
obtaining a plurality of sequence reads from a first sample obtained from an asymptomatic carrier of the pathogenic bacterium and obtaining a plurality of sequence reads from a second sample obtained from healthy individual;
using a method according to any one of claims 1 to 14 to obtain indications of the relative abundances of lineages and/or reference genomes within the first sample and the second sample;
comparing the indications of the relative abundances of the lineages and/or reference genomes within the first sample with the relative abundance of the lineages and/or reference genomes in the second sample;
selecting a bacterium with a genome, or belonging to a lineage, which is present in the first sample but absent in the second sample for bacteriotherapy for the disease.

28. A bacterium identified using a method according to any one of claims 24 to 27, wherein the bacterium is for use in a method of treating a dysbiosis or disease in an individual.

29. A bacterium for use in a method of treating a dysbiosis or disease in a patient, the method comprising identifying the bacterium using a method according to any one of claims 24 to 27, and administering the identified bacterium to the patient.

30. A method of treating a dysbiosis or disease in an individual, the method comprising identifying the bacterium using a method according to any one of claims 24 to 27, and administering a therapeutically effective amount of the identified bacterium to the patient.

31. A method of diagnosing a disease in a patient, the method comprising:
obtaining a plurality of sequence reads from a sample obtained from the patient;
using a method according to any one of claims 1 to 14 to obtain indications of the relative abundances of lineages and/or reference genomes within the sample;
comparing the indications of the relative abundances of the lineages and/or reference genomes within the sample with the relative abundance of the lineages and/or reference genomes in a control;
identifying a bacterium with a genome, or belonging to lineage, which is present in the sample obtained from the patient but absent from the control, or which is present at a higher abundance in the sample obtained from the patient compared with the control;
wherein the presence, or higher abundance, of the bacterium in the sample is indicative of the disease.

32. A method according to claim 31, wherein the method further comprises:
selecting the individual for treatment for the disease; or
subjecting an individual for treatment for the disease.

33. A diagnostic system for use in a method according to claim 31 or 32, the system comprising a tool or tools obtaining a plurality of sequence reads from a sample obtained from a patient; and
a computer programmed to compute indications of the relative abundances of lineages and/or reference genomes within the sample using a method according to any one of claims 1 to 14 from the sequencing data.

34. A method of treating a disease in a patient, the method comprising:
(i) requesting a test providing the results of an analysis, the test including:
obtaining a plurality of sequence reads from a sample obtained from the patient;
using a method according to any one of claims 1 to 14 to obtain indications of the relative abundances of lineages and/or reference genomes within the sample;
comparing the indications of the relative abundances of the lineages and/or reference genomes within the sample with the relative abundance of the lineages and/or reference genomes in a control;
identifying a bacterium with a genome, or belonging to lineage, which is present in the sample obtained from the patient but absent from the control, or which is present at a higher abundance in the sample obtained from the patient compared with the control;
wherein the presence, or higher abundance, of the bacterium in the sample is indicative of the disease;
(ii) treating the individual for the disease.

35. A method of identifying the bacterial causative agent of a disease in a patient, the method comprising:
obtaining a plurality of sequence reads from a sample obtained from a patient;
using a method according to any one of claims 1 to 14 to obtain indications of the relative abundances of lineages and/or reference genomes within the sample;
comparing the indications of the relative abundances of the lineages and/or reference genomes within the sample with the relative abundance of the lineages and/or reference genomes in a control;
identifying a bacterium with a genome, or belonging to lineage, which is present in the sample obtained from the patient but absent from the control, or which is present at a higher abundance in the sample obtained from the patient compared with the control;
wherein said bacterium is the causative agent of the disease.

36. A method analysing the bacteria and/or bacterial lineages present in a sample, wherein the method includes:
performing whole genome shotgun sequencing of (i) DNA extracted from a first portion of the sample and (ii) DNA extracted from bacteria cultured from a second portion of the sample using a bacterial culturing method;
identifying one or more reference genomes and/or lineages in a database to which at least one of the plurality of sequence reads obtained in (i) is deemed to uniquely map, wherein the database stores a plurality of reference genomes and phylogenetic information which relates the stored reference genomes to each other in a phylogenetic structure;
identifying one or more reference genomes and/or lineages in the database to which at least one of the plurality of sequence reads obtained in (ii) is deemed to uniquely map;
comparing the one or more reference genomes and/or lineages to which at least one of the plurality of sequence reads obtained in (i) were deemed to uniquely map with the one or more reference genomes and/or lineages to which at least one of the plurality of sequence reads obtained in (ii) were deemed to uniquely map.

37. A method according to claim 36 comprising:
identifying all reference genomes and/or lineages in a database to which at least one of the plurality of sequence reads obtained in (i) is deemed to uniquely map; and
identifying all reference genomes and/or lineages in the database to which at least one of the plurality of sequence reads obtained in (ii) is deemed to uniquely map.

38. A method according to claim 36 or 37, wherein the method is a method of determining the bacteria and/or bacterial lineages present in a sample which have or have not been cultured using a bacterial culturing method, wherein the method includes:
determining the bacteria and/or bacterial lineages present in the first portion of the sample which were and/or were not cultured using the bacterial culturing method by comparing the one or more reference genomes and/or lineages to which at least one of the plurality of sequence reads obtained in (i) were deemed to uniquely map with the one or more reference genomes and/or lineages to which at least one of the plurality of sequence reads obtained in (ii) were deemed to uniquely map.

39. A method according to claim 36 or 37, further comprising:
performing whole genome shotgun sequencing of (iii) DNA extracted from bacteria cultured from a second sample, obtained from the same source as the sample in (i) and (ii), using an alternate bacterial culturing method;
identifying one or more reference genomes and/or lineages in the database to which at least one of the plurality of sequence reads obtained in (iii) is deemed to uniquely map;
comparing the one or more reference genomes and/or lineages to which at least one of the plurality of sequence reads obtained in (iii) were deemed to uniquely map with the one or more reference genomes and/or lineages to which at least one of the plurality of sequence reads obtained in (i) were deemed to uniquely map and, optionally, the one or more reference genomes and/or lineages to which at least one of the plurality of sequence reads obtained in (ii) were deemed to uniquely map.

40. A method according to claim 39, wherein the method is a method of determining the bacteria and/or bacterial lineages present in a sample which have been cultured using an alternate bacterial culturing method, wherein the method includes:
(a) determining the bacteria and/or bacterial lineages present in the sample which were and/or were not cultured using the alternate bacterial culturing method by comparing the one or more reference genomes and/or lineages to which at least one of the plurality of sequence reads obtained in (iii) were deemed to uniquely map with the one or more reference genomes and/or lineages to which at least one of the plurality of sequence reads obtained in (i) were deemed to uniquely map; or
(b) determining the bacteria and/or bacterial lineages present in the sample which were cultured using the alternate bacterial culturing method and which were not cultured with the bacterial culturing method by comparing the one or more reference genomes and/or lineages to which at least one of the plurality of sequence reads obtained in (iii) were deemed to uniquely map with the one or more reference genomes and/or lineages to which at least one of the plurality of sequence reads obtained in (ii) were deemed to uniquely map and the one or more reference genomes and/or lineages to which at least one of the plurality of sequence reads obtained in (i) were deemed to uniquely map.

41. A method according to claim 36 or 37, wherein the method is a method of preparing a culture collection of bacteria of interest present in a sample, the method including identifying a bacterial culturing method for culturing the bacteria of interest, wherein the method includes:
determining the bacteria of interest present in the sample which were cultured using the bacterial culturing method by comparing the one or more reference genomes and/or lineages to which at least one of the plurality of sequence reads obtained in (i) were deemed to uniquely map with the one or more reference genomes and/or lineages to which at least one of the plurality of sequence reads obtained in (ii) were deemed to uniquely map; and
employing the bacterial culturing method to prepare a collection of cultures of said bacteria of interest from the sample.

42. A method according to claim 36 or 37, wherein the method is a method of obtaining the genomic sequence of one or more bacteria of interest present in a sample, wherein the method includes:
determining the bacteria of interest present in the sample which were cultured using the bacterial culturing method by comparing the one or more reference genomes and/or lineages to which at least one of the plurality of sequence reads obtained in (i) were deemed to uniquely map with the one or more reference genomes and/or lineages to which at least one of the plurality of sequence reads obtained in (ii) were deemed to uniquely map;
employing the bacterial culturing method to prepare cultures of one or more of the bacteria of interest from the sample, and
determining the genomic sequence(s) of said bacteria.

43. A method according to claim 42, further comprising adding the genomic sequence(s) of said one or more of the bacteria of interest to a database that stores reference genomes.

44. A method substantially as described herein with reference to any embodiment shown in the accompanying drawings.
